(19) United States
(12) Patent Application Publication
(10) Pub. No.: US 2004/0228842 A1
(43) Pub. Date: Nov. 18, 2004

(54) Compositions and methods for cytomegalovirus treatment

(76) Inventors: Shan Lu, Franklin, MA (US); Siyuan Shen, Worcester, MA (US)

Correspondence Address: FISH & RICHARDSON PC, 225 FRANKLIN ST, BOSTON, MA, 02110, US

(21) Appl. No.: 10/789,247

(22) Filed: Feb. 27, 2004

Related U.S. Application Data
(60) Provisional application No. 60/450,818, filed on Feb. 27, 2003.

(51) Int. Cl. A61K 48/00

(52) U.S. Cl. 424/93.2; 514/44

(57) ABSTRACT

A panel of nucleic acids encoding human cytomegalovirus (HCMV) antigens is disclosed. These include nucleic acids encoding any one or more of the following antigenic proteins: glycoprotein B (gB), glycoprotein M (gM), glycoprotein N (gN), glycoprotein complex II (gM/N), glycoprotein complex III, phosphoprotein pp65 (pp65), phosphoprotein pp150 (pp150), and antigenic fragments of any of these. These nucleic acids can be used in kits and methods of treating HCMV infections.

### CROSS-REFERENCE TO RELATED APPLICATIONS

This application claims the benefit of priority of U.S. Ser. No. 60/450,818, filed Feb. 27, 2003, the contents of which are hereby incorporated by reference in its entirety.

### TECHNICAL FIELD

This invention relates to compositions and methods of inhibiting and treating cytomegalovirus (CMV) infection using nucleic acid compositions.

### BACKGROUND

Human cytomegalovirus (HCMV) is a member of the family of herpes viruses. HCMV is endemic within the human population and infection rarely causes symptomatic disease in immunocompetent individuals. However, HCMV infection of immunocompromised patients, including AIDS patients and organ transplant recipients, can have serious consequences. HCMV infection has been shown to cause a variety of disorders in the immunocompromised, including pneumonitis, retinitis, disseminated viremia, and organ dysfunction. HCMV also poses a serious threat to the health of HIV-positive individuals because HCMV may accelerate the development of AIDS as well as contribute to the morbidity associated with increased immunodeficiency. Likewise, HCMV infection can be problematic for pregnant women and children, especially infants and newborns (Castillo and Kowalik, Gene, 290:19-34 (2002), Britt and Alford, Fields Virology, Third Ed. Chapter 77, pp. 2493-2523 (1996)). HCMV infection of newborns and the immunocompromised can be fatal.

Live attenuated vaccines have been used in the treatment of and protection against HCMV. However, due to their ineffectiveness, a new strategy is needed in designing a vaccine for HCMV.

### SUMMARY

The invention features nucleic acid (e.g., naked DNA plasmid) CMV (e.g., HCMV) compositions that can elicit a broad immune response in the recipient. Specifically, neutralizing antibody responses, or both a neutralizing antibody immune response and a cell-mediated immune response can be induced by immunization with one of more of the CMV constructs of this panel. These compositions, or constructs, include nucleic acids (e.g., naked DNA plasmids) encoding antigenic proteins of CMV (e.g., HCMV). Specifically, these antigenic proteins can include glycoprotein B (gB)(also referred to as glycoprotein complex I (gcI)), glycoprotein M (gM), glycoprotein N (gN), glycoprotein complex II (gcII or gM/N or gM/gN), glycoprotein complex III (gcIII or gH/L/O), phosphoprotein 65 (pp65), phosphoprotein 150 (pp150), and/or antigenic fragments (e.g., peptides) of any of these proteins. Once administered to a recipient, the compositions cause cells in a recipient to produce antigenic proteins and peptides, which elicit a neutralizing antibody immune response, or both a neutralizing antibody immune response as well as a cell-mediated immune response in the recipient.

In one aspect, the invention features compositions that include a plurality of sets of nucleic acid molecules, each set of nucleic acid molecules encoding a different type of CMV polypeptide, and each molecule of a set encoding the same type of CMV polypeptide, wherein one or more sets of the plurality encodes a CMV polypeptide that induces a neutralizing antibody response, and one or more sets of the plurality encodes a CMV polypeptide that induces a cell-mediated immune response.

In various embodiments, the nucleic acid molecules are nucleic acid vectors (e.g., plasmids) which include elements that promote expression of the CMV polypeptide, such as a promoter that is operably linked to the sequence encoding the CMV polypeptide (e.g., a CMV immediate early (IE) promoter, or another promoter that drives CMV gene expression in human cells), optionally, a leader peptide (e.g., a leader peptide which is expressed in frame with the CMV polypeptide, and which can replace a leader peptide encoded by the CMV gene), and a polyadenylation sequence (e.g., a bovine growth hormone polyA sequence).

In various embodiments, the CMV polypeptides encoded by the nucleic acids are human CMV polypeptides. The CMV polypeptides that induce an antibody response can be selected from the group of glycoprotein B (e.g., gB of HCMV), glycoprotein complex II (e.g., gN and gM of HCMV), glycoprotein complex III (e.g., gH, gL, and gO of HCMV), and antigenic fragments thereof. In certain embodiments, the CMV polypeptides that induce an antibody response are selected from glycoprotein B and glycoprotein complex II, or antigenic fragments thereof.

The CMV polypeptides that induce a cell-mediated immune response can be selected from the group of phosphoprotein pp65 (pp65) (e.g., HCMV pp65), phosphoprotein pp150 (pp150) (e.g., HCMV pp150), and antigenic fragments thereof.

In another aspect, the invention features compositions that includes nucleic acid vectors encoding a plurality of types of HCMV polypeptides that induce a neutralizing antibody response and optionally one or more nucleic acid vectors encoding one or more types of HCMV polypeptides that induce a cell-mediated immune response.

The invention also features compositions that include a plurality of sets of nucleic acid vectors encoding HCMV polypeptides, each set of nucleic acid vectors encoding a different type of HCMV polypeptide that induces a cell-mediated immune response. The compositions can further include a plurality of sets of nucleic acid vectors encoding HCMV polypeptides, each set of nucleic acid vectors encoding a different type of HCMV polypeptide that induces a neutralizing antibody response. The polypeptides that induce a neutralizing antibody response can be selected from the group of glycoprotein B, gM, gN, a combination of gM and gN (glycoprotein complex II; gcII), and a combination of gH, gL, and gO (glycoprotein complex III; gcIII) of HCMV, and antigenic fragments thereof. For example, the polypeptides that induce a cell-mediated immunity-inducing can include phosphoprotein 65, phosphoprotein 150, both phosphoprotein 65 and phosphoprotein 150, and/or antigenic fragments thereof.

The polypeptides that induce a neutralizing antibody response can include one of the following combinations: gcII or antigenic fragments thereof; gcIII or antigenic fragments thereof; gB and gcII or antigenic fragments thereof; gB and gcIII or antigenic fragments thereof.

In some aspects, the invention provides nucleic acid molecules that encode more than one CMV polypeptide (e.g., two, three, four, five, or six specific CMV polypeptides). Thus, the nucleic acid molecules of a single set can encode one or more CMV polypeptides that induce a neutralizing antibody response (e.g., gcII or antigenic fragments thereof, gcIII or antigenic fragments thereof) and/or one or more polypeptides that stimulate a cell-mediated immune response (e.g., pp65, pp150, or antigenic portions thereof).

In another aspect, the invention features compositions that include a plurality of sets of nucleic acid molecules, each set of nucleic acid molecules encoding a different type of CMV polypeptide that induces a neutralizing antibody response. In various examples, the CMV polypeptides include gcII or antigenic fragments thereof; gcIII or antigenic fragments thereof; gB and gcII or antigenic fragments thereof; and gB and gcIII of HCMV or antigenic fragments thereof.

The invention also features pharmaceutical compositions that elicit an immune response against HCMV. The compositions can include, for example, one or more of the nucleic acid compositions described herein and a pharmaceutically acceptable carrier. The nucleic acid compositions can include other features described herein.

The invention also features methods of and kits for eliciting an immune response against CMV (e.g., HCMV) in a subject. The methods include, for example, administering to the subject an amount of a pharmaceutical composition (e.g., a pharmaceutical composition described herein) effective to elicit an immune response against CMV in the subject. The immune response can be a protective immune response. The administration can be, e.g., by needle injection, needle-less jet injection, gene gun, topical administration, surgical administration, or mucosal administration. The subject can be a non-human mammal or a human (e.g., a human sero-negative for HCMV, e.g., a female between the ages of 11 and 40, a female contemplating pregnancy, a pregnant female, an HIV-infected individual, a future organ transplant recipient, and a future bone marrow donor, or, e.g., a human who is sero-positive for HCMV).

The new nucleic acid compositions (also referred to herein as DNA vaccines) have the advantages of having a combination of nucleic acid molecules that can elicit a strong immune response against HCMV. They encode polypeptides that elicit a neutralizing antibody immune response and/or nucleic acid molecules that encode polypeptides that elicit a cell-mediated immune response against CMV. Certain combinations of the nucleic acid compositions contain a combination of nucleic acids that when they express their encoded antigen or antigenic fragment can induce an unexpectedly strong neutralizing antibody response, e.g., in the combination of gM/N and the combination of gM/N with gB. The combinations used in the new compositions elicit greater immune responses as a combination than as individual components alone.

Thus, these nucleic acid compositions have the advantage of providing broad immune protection against CMV (e.g., HCMV) using different immune mechanisms. The combination of eliciting neutralizing antibody immune response and cell-mediated immune response confers advantages as a novel therapeutic agent for CMV (e.g., HCMV). Furthermore, combinations of certain antigens or antigenic fragments that unexpectedly provide a strong immune response when combined also confer advantages as a novel therapeutic agent for CMV (e.g., HCMV).

Exemplary amino acid and nucleic acid sequences for HCMV gB, gM, gN, gL, gH, gO, pp65, and pp150 are provided herein. In various embodiments, a nucleic acid composition includes a gB nucleic acid sequence according to SEQ ID NO:1, or a sequence that is at least 75%, 80%, 85%, 90%, 95%, 97%, 99%, or more identical to SEQ ID NO:1. A nucleic acid composition can encode a gB amino acid sequence that is 75%, 80%, 85%, 90%, 95%, 97%, 99%, or more identical to SEQ ID NO:2. The nucleic acid compositions can include a gM nucleic acid sequence that is at least 75%, 80%, 85%, 90%, 95%, 97%, 99%, or more identical to SEQ ID NO:3. The nucleic acid compositions can encode a gB amino acid sequence that is at least 75%, 80%, 85%, 90%, 95%, 97%, 99%, or more identical to SEQ ID NO:4.

A nucleic acid composition can include a sequence that is at least 75%, 80%, 85%, 90%, 95%, 97%, 99%, or more identical to a gN nucleic acid of SEQ ID NO:5, and/or the compositions can encode an amino acid sequence that is at least 75%, 80%, 85%, 90%, 95%, 97%, 99%, or more identical to SEQ ID NO:6.

The nucleic acid compositions can include a sequence that is at least 75%, 80%, 85%, 90%, 95%, 97%, 99%, or more identical to a gH nucleic acid sequence according to SEQ ID NO:7, or the nucleic acids can encode an amino acid sequences at least 75%, 80%, 85%, 90%, 95%, 97%, 99%, or more identical to SEQ ID NO:8.

The nucleic acid compositions can include a sequence that is at least 75%, 80%, 85%, 90%, 95%, 97%, 99%, or more identical to a gL nucleic acid sequence according to SEQ ID NO:9, and/or the compositions can encode an amino acid sequences at least 75%, 80%, 85%, 90%, 95%, 97%, 99%, or more identical to SEQ ID NO:10.

The nucleic acid compositions can include a sequence that is at least 75%, 80%, 85%, 90%, 95%, 97%, 99%, or more identical to a gO nucleic acid according to SEQ ID NO:11, and/or the nucleic acids can encode an amino acid sequence at least 75%, 80%, 85%, 90%, 95%, 97%, 99%, or more identical to SEQ ID NO:12.

The nucleic acid compositions can include a sequence that is at least 75%, 80%, 85%, 90%, 95%, 97%, 99%, or more identical to a pp65 nucleic acid of SEQ ID NO:13, and/or the sequences can encode an amino acid sequence at least 75%, 80%, 85%, 90%, 95%, 97%, 99%, or more identical to SEQ ID NO:14.

The nucleic acid compositions can include a sequence that is at least 75%, 80%, 85%, 90%, 95%, 97%, 99%, or more identical to a pp150 nucleic acid of SEQ ID NO:15, and/or the compositions can encode an amino acid sequence at least 75%, 80%, 85%, 90%, 95%, 97%, 99%, or more identical to SEQ ID NO:16.

The terms “gM/gN” or “gcII” refer to a nucleic acid composition containing a combination of nucleic acids encoding gM and gN. Thus “gM/gN” and “gcII” are used interchangeably herein.

The terms “gcIII” or “gH/L/O” refer to a nucleic acid composition containing a combination of nucleic acids encoding gH, gL, and gO, and are used interchangeably herein.

As used herein, “polypeptides” include both peptides and proteins. The term “CMV polypeptide” includes CMV polypeptides that are full-length polypeptides encoded by a CMV gene, or antigenic portions or fragments thereof.

As used herein, the term “substantially identical” (or “substantially homologous”) refers to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have at least 80% sequence identity. In the case of antibodies, the second antibody has the same specificity and has at least 50% of the affinity of the first antibody.

Calculations of “identity” between two sequences are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 50% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. The percent identity between two sequences is determined using the Needleman and Wunsch, J. Mol. Biol., 48:444-453, 1970, algorithm which has been incorporated into the GAP program in the GCG software package, using a Blossum 62 scoring matrix with a gap penalty of 12, a gap extend penalty of 4, and a frameshift gap penalty of 5.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. 6.3.1-6.3.6, 1989, which is incorporated herein by reference. Aqueous and nonaqueous methods are described in that reference and either can be used. Specific hybridization conditions referred to herein are as follows: 1) low stringency hybridization conditions: 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions: 6×SSC at about 45° C., followed by one or more washes in 0.2×SSC, 0.1% SDS at 60° C.; 3) high stringency hybridization conditions: 6×SSC at about 45° C., followed by one or more washes in 0.2×SSC, 0.1% SDS at 65° C.; and 4) very high stringency hybridization conditions: 0.5 M sodium phosphate, 7% SDS at 65° C., followed by one or more washes at 0.2×SSC, 1% SDS at 65° C.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the description and drawings, and from the claims.

### BRIEF DESCRIPTION OF THE DRAWINGS

FIG. 1 is a schematic depiction of the different antigenic proteins encoded by nucleic acid constructs that can be used in a vaccine against human cytomegalovirus (HCMV). Each bar represents the length of the encoded amino acid sequence. DNA fragments of these different genes were subcloned into an optimized mammalian expression vector pJW4303, which contains the CMV promoter. “gB” is the wild-type glycoprotein B protein. “gBs” is the wild-type glycoprotein B protein truncated at its carboxy terminus from 907 amino acids to 692 amino acids, thus removing its transmembrane domain. “tPA.gBs” is the carboxyterminal truncation of glycoprotein B containing the tPA leader sequence. “gM” is the wild-type glycoprotein M protein which is 372 amino acids in length. “gN” is the wild-type glycoprotein N protein which is 138 amino acids in length. “pp65” is the wild-type phosphoprotein protein which is 561 amino acids in length. “pp150” is the wild-type phosphoprotein protein which is 1048 amino acids in length. These antigenic proteins can be present in a number of different combinations or individually for inducing immune responses.

FIG. 2 is a graph showing that antisera raised by vaccination of rabbits with gB and gBs have similar efficacy in neutralizing HCMV AD169 virus (100 pfu). Both constructs induced neutralizing antibody response to AD169 viral infection as seen by the percent reduction of infection. The x-axis is antisera dilutions from 1 to 1024 and the y-axis is the percent reduction of HCMV infection as measured by reduction of the infected CV-1 cell nuclei.

FIGS. 3A and 3B are western blots showing recognition of gM (FIG. 3A) or gN (FIG. 3B) in 293T cells transfected with the following DNA vaccines: gM+gN (lane3), gM (lane 4) or gN (lane 5). CMV virion samples grown from FSK cells (lane 1) were used as positive controls and uninfected FSK cells (lane 2) or 293T cells transfected with empty DNA vaccine vector (lane 6) were included as the negative controls. Mouse monoclonal antibodies against gM (FIG. 3A) or gN (FIG. 3B) were used to test the expression of these DNA vaccines.

FIG. 4 is a western blot showing the specificity of anti-gM/gN antibody responses in sera from rabbits immunized with the gM+gN DNA vaccine. Lane 1 contains lysate of HCMV infected FSK cells (positive control). Lane 2 contains lysate of non-infected FSK cells (negative control). Lane 3 contains lysate from 293T cells transfected with gM+gN. Lane 4 contains lysate from 293T cells transfected with gM. Lane 5 contains lysate from 293T cells transfected with gN. Lane 6 contains lysate from cells tranfected with empty vector (negative control).

FIG. 5 is a western blot showing specific antigen (lane a) recognition by a 1:100 fold dilution of serum from immunized Balb/C mice. The clean background seen in the control lane (lane b) suggests that the immune response to pp65 is highly specific.

FIGS. 6A-D are a series of plots of a FACS result of intracellular staining (ICS) analysis of splenocytes from mice immunized with pp65 naked DNA plasmid vaccine. The first three plots, FIGS. 6A-C, are results of a FACS analysis of splenocytes from a mouse immunized with a pp65 vaccine. The fourth plot, FIG. 4D, is the negative control showing the results of a FACS analysis of splenocytes from a mouse immunized with vector control alone. CD8 expression is plotted on the x-axis. IFN-γ expression is plotted on the y-axis.

FIGS. 7A-D are a series of plots of a FACS result of ICS analysis of splenocytes from mice immunized with pp150 naked DNA plasmid vaccine. The first three plots, FIGS. 7A-C, are results of a FACS analysis of splenocytes from a mouse immunized with the pp150 DNA vaccine. The fourth plot, FIG. 7D, is the negative control showing the results of splenocytes from a mouse immunized with vector control alone. CD8 expression is plotted on the x-axis. IFN-γ expression is plotted on the y-axis.

FIG. 8A is a schematic depiction of the different gcIII antigenic proteins encoded by nucleic acid constructs that can be used in a vaccine against human cytomegalovirus (HCMV). Each bar represents the length in amino acid sequence encoded. DNA fragments of these different genes were subcloned into an optimized mammalian expression vector pJW4303, which contains the CMV promoter. “gH” is the wild-type glycoprotein H protein which is 743 amino acids in length. “gL” is the wild-type glycoprotein L which is 278 amino acids in length. “gO” is the wild-type glycoprotein O protein which is 466 amino acids in length. These antigenic proteins can be present in a number of different combinations or individually for inducing immune responses.

FIG. 8B is a western blot showing the recognition of the gH protein in various samples. Lane 1 contains lysate of HCMV infected FSK cells (positive control). Lane 2 contains lysate of non-infected FSK cells (negative control). Lane 3 contains lysate from 293T cells transfected with gH. Lane 4 contains lysate from 293T cells transfected with gH+gL. Lane 5 contains lysate from 293T cells transfected with gH+gL+gO. Lane 6 contains lysate from 293T cells tranfected with gH+gL+gB. Lane 7 contains lysate from 293T cells transfected with gH+gL+gO+gB. Lane 8 contains lysate from 293T cells transfected with empty vector (negative control).

FIG. 8C is a western blot showing the recognition of the gL protein in various samples. Lane 1 contains lysate of HCMV infected FSK cells (positive control). Lane 2 contains lysate of non-infected FSK cells (negative control). Lane 3 contains lysate from 293T cells transfected with gH. Lane 4 contains lysate from 293T cells transfected with gH+gL. Lane 5 contains lysate from 293T cells transfected with gH+gL+gO. Lane 6 contains lysate from 293T cells tranfected with gH+gL+gB. Lane 7 contains lysate from 293T cells transfected with gH+gL+gO+gB. Lane 8 contains lysate from 293T cells transfected with empty vector (negative control).

FIGS. 9A and 9B are a set of western blots showing recognition of the gH (FIG. 9A, upper panel) and gL (FIG. 9B, lower panel) proteins in various sample. “L” and “S” directly under each lane refer to samples from cell lysates and supernatants, respectively. gH/L/O refers to samples in which gH, gL, and gO were co-expressed. gH/gL refers to samples from cells in which gH and gL were co-expressed. gH/O refers to samples from cells in which gH and gO were co-expressed. gH refers to a sample from cells in which gH was expressed. Vec refers to a sample in which empty vector was transfected.

FIG. 10 is a representation of a HCMV gB nucleic acid sequence (SEQ ID NO:1).

FIG. 11 is a representation of a HCMV gB amino acid sequence (SEQ ID NO:2).

FIG. 12 is a representation of HCMV gM nucleic acid and amino acid sequences (SEQ ID NO:3 and SEQ ID NO:4, respectively).

FIG. 13 is a representation of HCMV gN nucleic acid and amino acid sequences (SEQ ID NO:5 and SEQ ID NO:6, respectively).

FIG. 14 is a representation of a HCMV gH nucleic acid sequence (SEQ ID NO:7).

FIG. 15 is a representation of a HCMV gH amino acid sequence (SEQ ID NO:8).

FIG. 16 is a representation of HCMV gL nucleic acid and amino acid sequences (SEQ ID NO: 9 and SEQ ID NO:10, respectively).

FIG. 17 is a representation of HCMV gO nucleic acid and amino acid sequences (SEQ ID NO:11 and SEQ ID NO:12, respectively).

FIG. 18 is a representation of HCMV pp65 nucleic acid and amino acid sequences (SEQ ID NO:13 and SEQ ID NO:14, respectively).

FIG. 19 is a representation of a HCMV pp150 nucleic acid sequence (SEQ ID NO:15).

FIG. 20 is a representation of a HCMV pp150 amino acid sequence (SEQ ID NO:16).

Like reference symbols in the various drawings indicate like elements.

### DETAILED DESCRIPTION

The invention provides a panel of nucleic acids that can be used to inhibit or treat CMV (e.g., HCMV). This panel includes nucleic acid compositions (e.g., naked DNA plasmid vectors) encoding antigenic proteins of HCMV (e.g., of strain AD169). Specifically, these antigenic proteins can include glycoprotein B (gB or also referred to as glycoprotein complex I (gcI)), glycoprotein M (gM), glycoprotein N (gN), a combination of gM and gN called glycoprotein complex II (gcII or gM/N or gM/gN), a combination of gH, gL, and gO called glycoprotein complex III (gcIII or gH/L/O), phosphoprotein pp65 (pp65), phosphoprotein pp150 (pp150), or antigenic fragments (e.g., peptides) of any of these. Antigenic proteins of HCMV can include those that induce a neutralizing antibody immune response (e.g., gB, gM, gN, gcII, and gcIII). Antigenic proteins of HCMV also include those that induce a cell-mediated immune response (e.g., pp65 and pp150). The new vaccines can include DNA encoding more than one of these antigenic proteins or fragments.

For example, the compositions can include nucleic acid molecules (e.g., naked DNA plasmids) encoding gcII (gM/gN) and/or gcIII (gH/L/O). In other examples, the compositions can include nucleic acid molecules encoding gB in combination with either or both nucleic acid molecules encoding gcII and nucleic acid molecules encoding gcIII. Alternatively, the compositions can include nucleic acid molecules encoding gB, gcII, and pp65. Another example is a composition including nucleic acid molecules encoding gB, gcII, and pp150. In yet another example, a composition can include nucleic acid molecules encoding gB, gcII, pp65, and pp150. Any number of combinations of DNA encoding polypeptide capable of inducing neutralizing antibody with or without DNA plasmid (e.g., naked DNA plasmid) encoding polypeptide capable of inducing cell-mediated immune response can be used for the CMV (e.g., HCMV) vaccines of the invention.

The new nucleic acid compositions can induce a protective immune response in an individual (i.e., protection against a subsequent exposure to HCMV that inhibits or prevents an HCMV infection). This immune response can include humoral immunity or cell-mediated immunity, or both. Humoral immunity involves the induction of neutralizing antibodies against the infecting organism (e.g., CMV, e.g., HCMV). Cell-mediated immunity involves a cytotoxic T lymphocyte response to the infecting organism (e.g., CMV, e.g., HCMV). Humoral immunity can be induced by gB, gcII, gcIII, or any combination of these, antigenic fragments of these, or antigenic fragment combinations of these. Cell-mediated immunity can be elicited by pp65 and/or pp150 or antigenic fragments of these.

The new nucleic acid compositions combine antigens that elicit neutralizing antibody immune response to CMV (e.g., HCMV) and antigens that elicit cell-mediated immune response to CMV (e.g., HCMV) in a single composition. Administration of any one of these compositions can include a prime or boost with attenuated virus vaccine and/or polypeptide vaccine, or may not include either of these supplements.

Human Cytomegalovirus Proteins

The invention is based, in part, on the discovery that administering nucleic acids encoding HCMV proteins in particular combinations promotes expression of the proteins and can enhance immune responses to the virus. Expressing these combinations can stimulate both humoral and cell-mediated immune responses, thus providing potent broad-spectrum immunity. Providing HCMV glycoprotein expression via DNA administration has the added benefit of allowing biosynthesis and assembly of the proteins in human cells, and eliminates complications of glycoprotein expression and purification ex vivo. Because the glycoproteins provided herein can be expressed within the cells of a subject, they can include membrane-associated domains, glycosylation, and other post-translational modifications which confer immunogenic properties on the polypeptides.

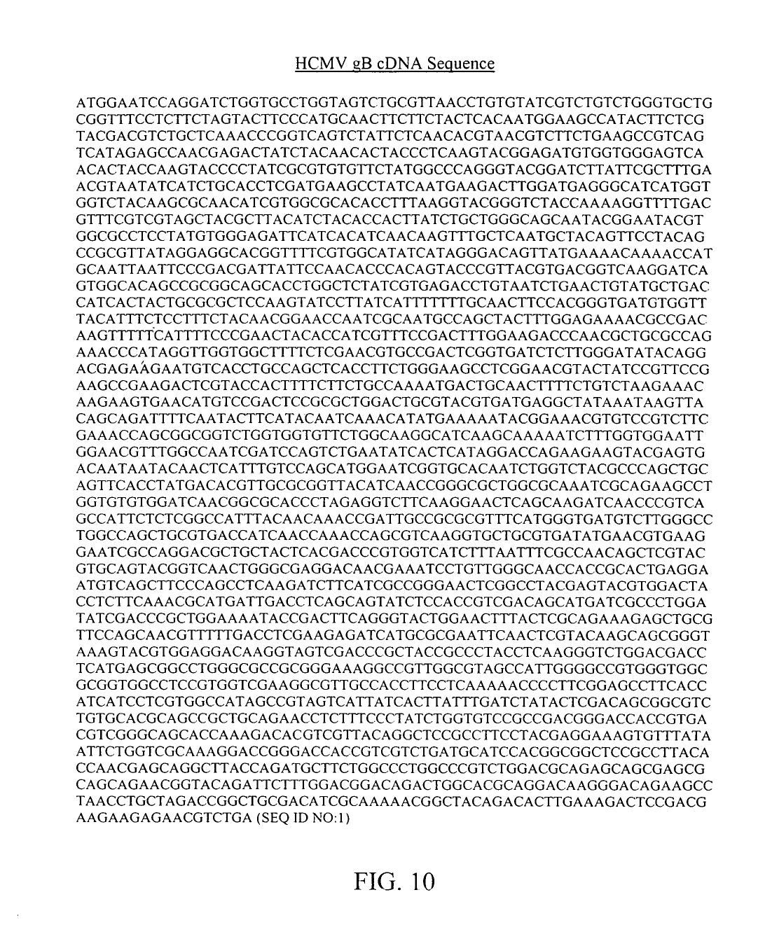

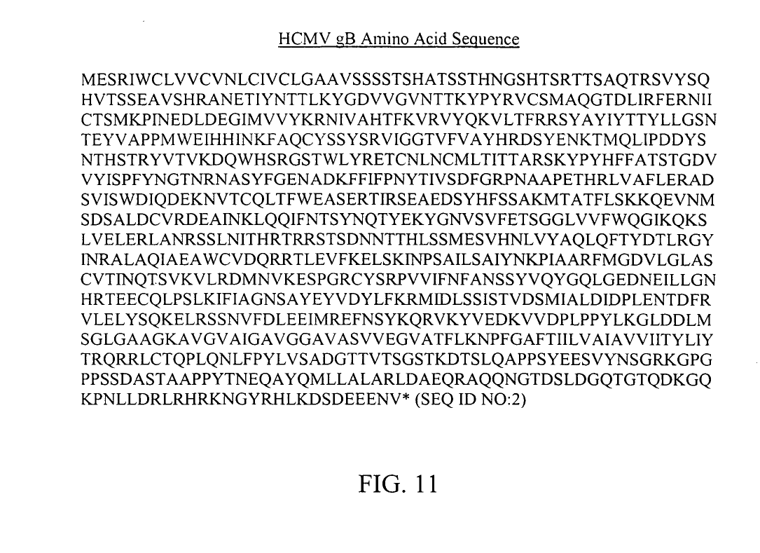

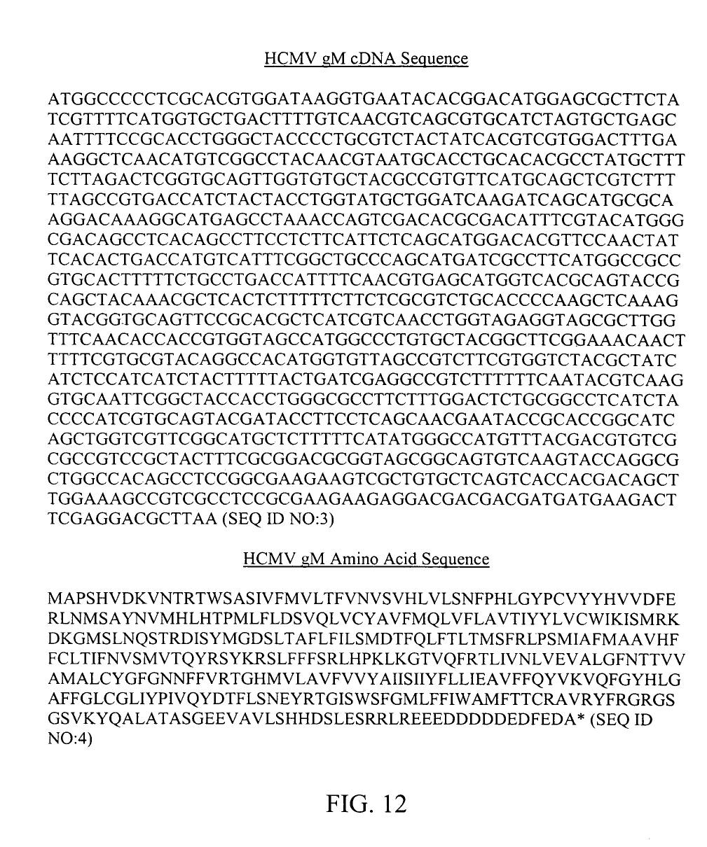

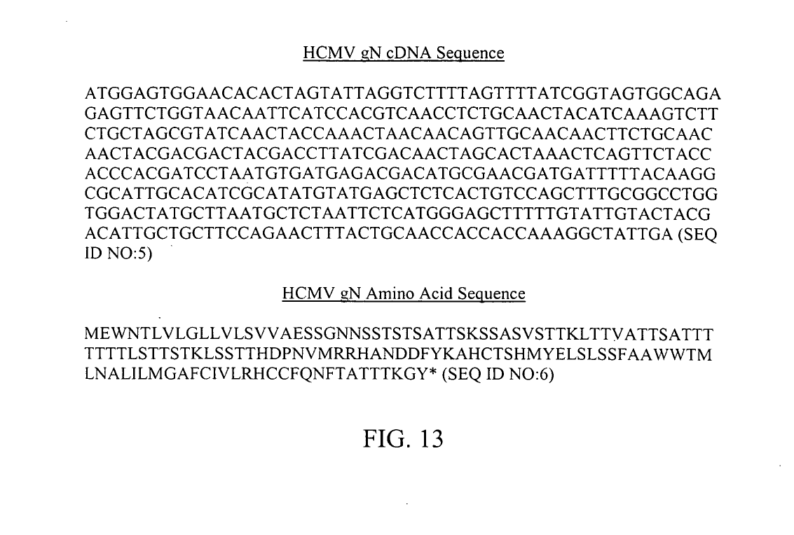

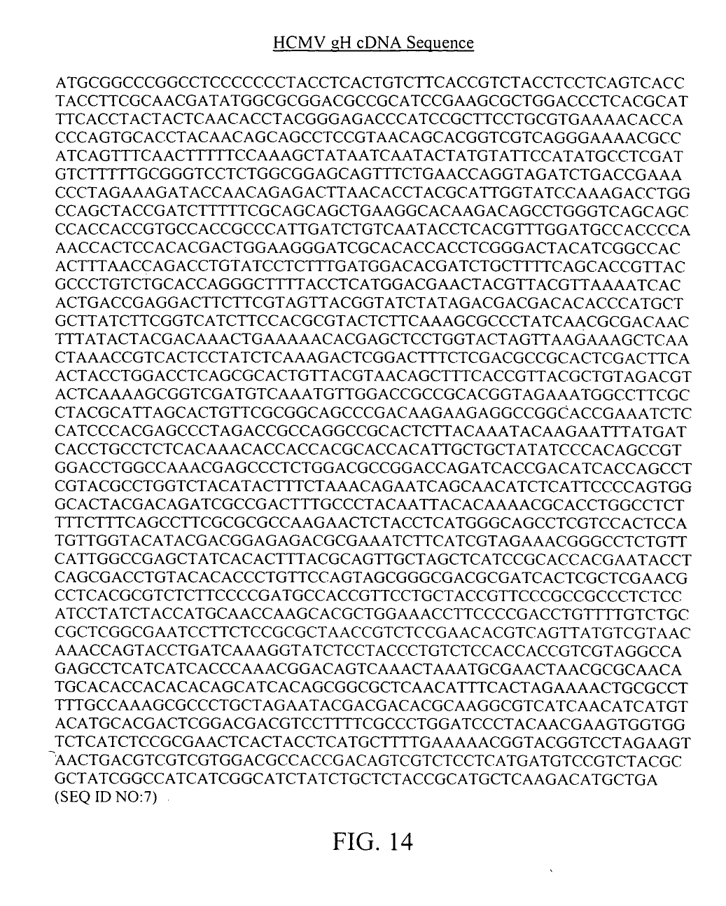

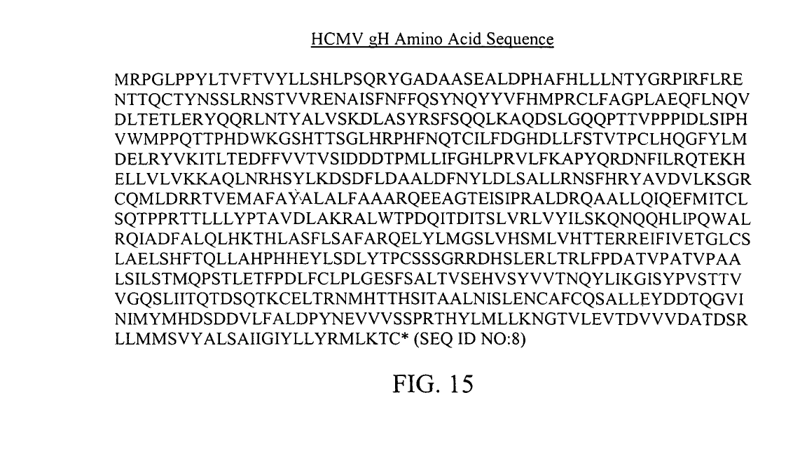

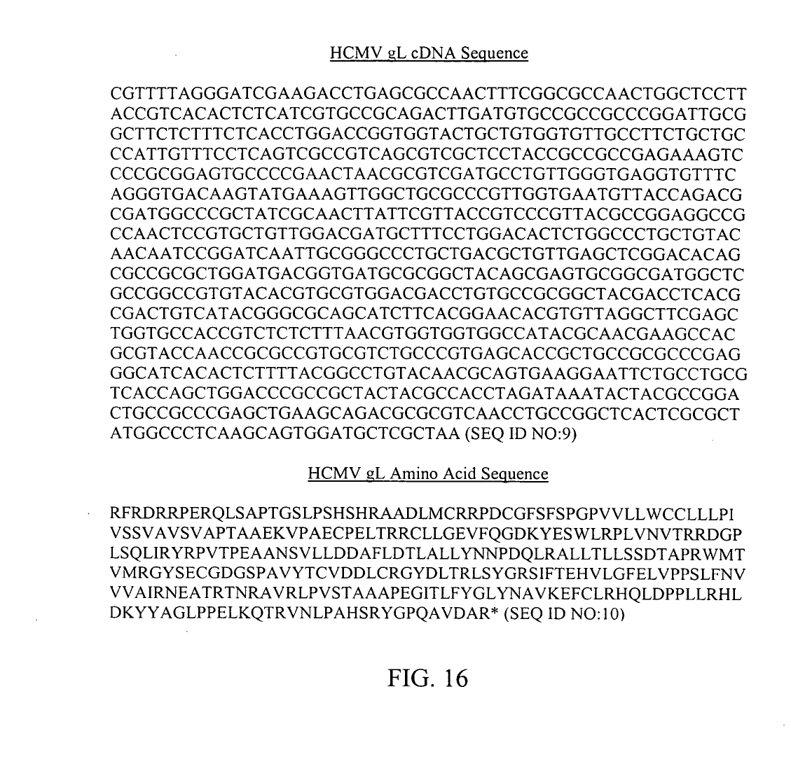

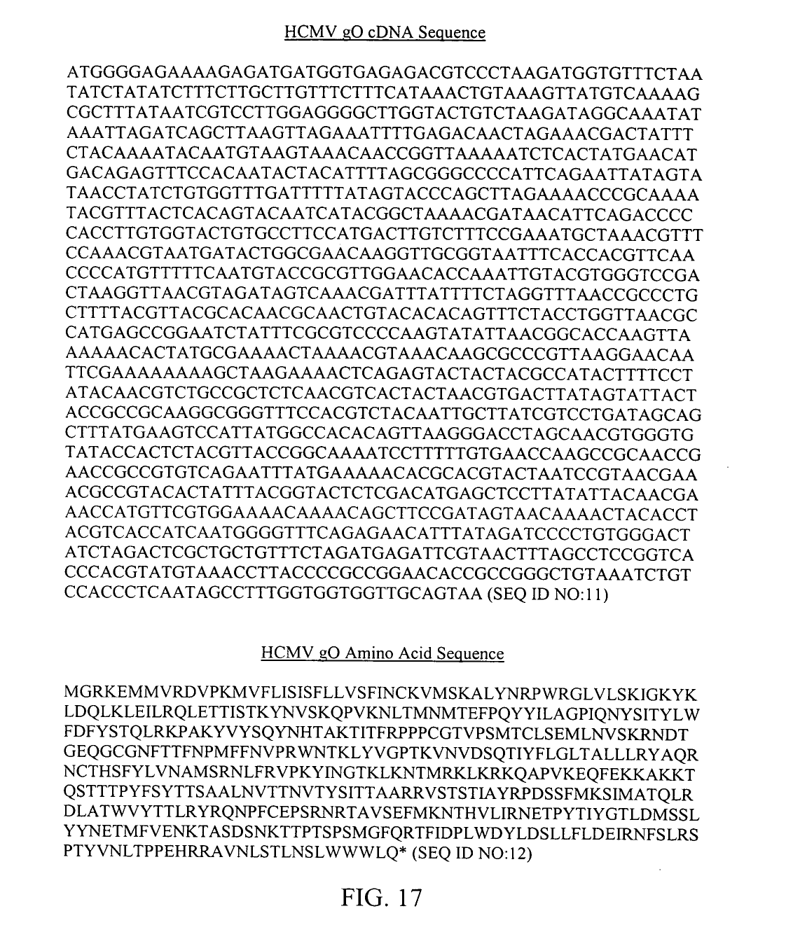

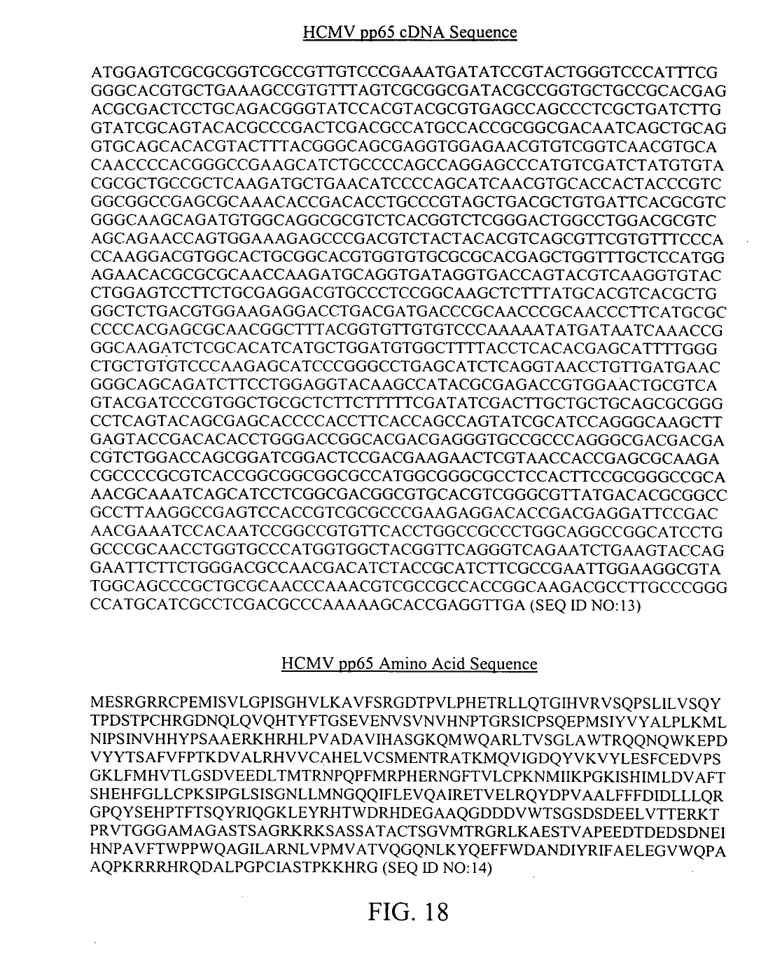

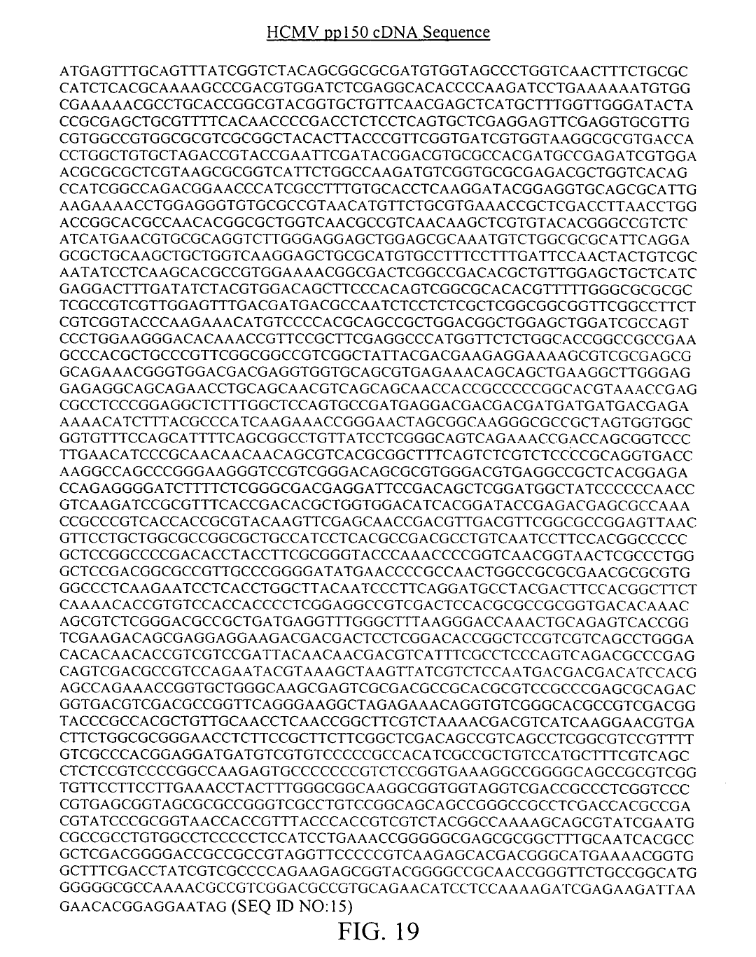

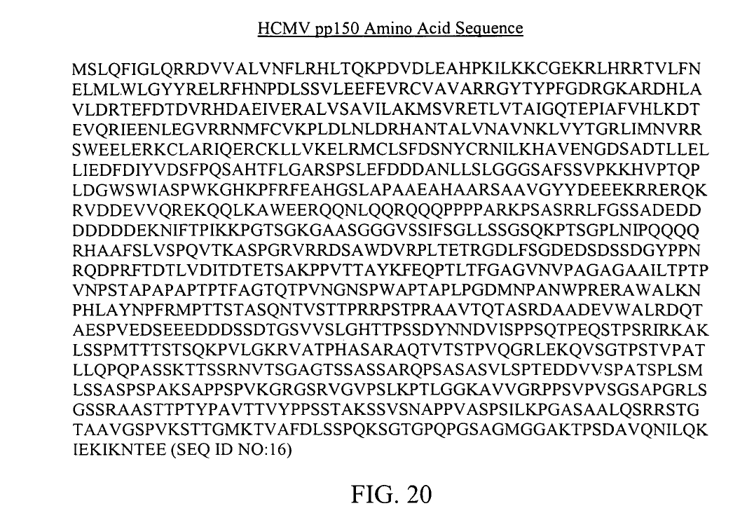

Exemplary nucleic acid and amino acid sequences of HCMV gN, gM, gL, gH, gO, gB, pp65, and pp150 are provided herein (see, e.g., FIGS. 10-20, and SEQ ID NOs:1-16). The compositions described herein can include and/or encode sequences that are at least 75%, 80%, 85%, 90%, 95%, 97%, 99%, or more identical to one or more of SEQ ID NOs:1-16. In addition, or alternately, the compositions described herein can include sequences that are from a laboratory-adapted strain (e.g., AD169, Towne) or a natural isolate. Natural isolates can be derived from infected patients by methods known in the art (see, e.g., Walker et al., J. Clin. Microbio., 39:2219-2226,2001 and references cited therein for discussion of sequence variability and method of isolating and comparing HCMV strains).

The nucleic acid compositions herein can encode full-length HCMV gene products, or may include antigenic fragments thereof. Antigenic fragments are fragments that are immunogenic when administered to an animal (e.g., under conditions that promote an immune response, e.g., with an adjuvant, or, e.g., when expressed from a DNA vaccine). Antigenic fragments are at least 10, 20, or 30 amino acids in length, or represent a specific domain (e.g., a soluble portion of a membrane glycoprotein). Methods for determining whether a fragment is immunogenic are known. In one example, a nucleic acid composition encoding a fragment of a HCMV protein is administered to an animal, followed by 2-4 boosts of the nucleic acid at periods of 2-4 weeks thereafter. Serum from the animal is isolated and tested for reactivity with the protein encoded by the nucleic acid (e.g., reactivity with a transfected cell lysate by western blot). Methods for testing cell-mediated immunity are also known, and include assays to measure cytokine release and/or proliferation of lymphocytes in response to stimulation with antigen.

Nucleic Acid Compositions

Nucleic acid compositions are useful in inhibiting, e.g., preventing, or controlling infection or disease by inducing immune responses, to an antigen or antigens, in an individual. For example, nucleic acid compositions can be used prophylactically in naive individuals, or therapeutically in individuals already infected with CMV (e.g., HCMV). Traditional vaccines, which include inactivated viruses or subunit protein or peptide antigen, have had poor immunogenicity and poor induction of cell-mediated immunity, and low efficacy. In addition, live attenuated vaccines can have safety concerns. A major advantage of nucleic acid-based compositions is that they can induce both antibody and cell-mediated immune responses. The development of nucleic acid vaccines has proved to be promising, and they can be administered in combination with or as a boost to traditional inactivated virus and/or subunit protein or peptide antigen.

The nucleic acid compositions described herein have the advantage of containing a combination of nucleic acid molecules that encode polypeptides that elicit neutralizing antibody immune response and nucleic acid molecules that encode polypeptides that elicit cell-mediated immune response against CMV (e.g., HCMV). Various combinations of nucleic acids described herein elicit greater immune responses in combination than as individual components alone. In certain embodiments, the combinations can provide a greater than additive (or synergistic) effect. Thus, these compositions have the advantage of providing broad immune protection against CMV, (e.g., HCMV) using different immune mechanisms. This combination of eliciting neutralizing antibody immune response and cell-mediated immune response confers advantages for inhibition of CMV (e.g., HCMV).

Nucleic acid compositions for inducing immune responses can consist of naked DNA plasmids that express the antigen. Bacterial vectors, replicon vectors, live attenuated bacteria, DNA vaccine co-delivery with live attenuated vectors, and viral vectors for expression of heterologous genes also can be used. Bacterial vectors such as BCG and Listeria have been used and show promise. In the case of naked DNA replicon vectors, a mammalian expression plasmid serves as a vehicle for the initial transcription of the replicon.

The replicon is amplified within the cytoplasm, resulting in more abundant mRNA encoding the heterologous gene such that initial transfection efficiency may be less important for immunogenicity. Live attenuated viral vectors (e.g., recombinant vaccinia (e.g., modified vaccinia Ankara (MVA), IDT Germany), recombinant adenovirus, avian poxvirus (e.g., canarypox (e.g., ALVAC®, Aventis Pasteur) or fowlpox), poliovirus, and alphavirus virion vectors) have been successful in inducing cell-mediated immune response and can be used as well. The avian poxviruses are defective in mammalian hosts, but can express inserted heterologous genes under early promoters. Recombinant adenovirus and poliovirus vectors can thrive in the gut and so can stimulate efficient mucosal immune responses. Finally, attenuated bacteria as a vehicle for DNA vaccination delivery has been useful. Examples include S. enterica, S. tymphimurium, Listeria, and BCG. The use of mutant bacteria with weak cell walls can aid the exit of DNA plasmid from the bacterium.

DNA uptake can sometimes be improved by the use of the appropriate adjuvants. Synthetic polymers (e.g., polyamino acids, co-polymers of amino acids, saponin, paraffin oil, muramyl dipeptide, Regressin (Vetrepharm, Athens Ga.), and Avridine) and liposomal formulations can be added as adjuvants to the vaccine formulation to improve DNA stability and DNA uptake by the host cells, and may decrease the dosage required to induce an effective immune response. Regardless of route, adjuvants can be administered before, during, or after administration of the nucleic acid. Not only can the adjuvant increase the uptake of nucleic acid into host cells, it can increase the expression of the antigen from the nucleic acid within the cell, induce antigen presenting cells to infiltrate the region of tissue where the antigen is being expressed, or increase the antigen-specific response provided by lymphocytes.

Nucleic acid uptake can be improved in other ways as well. For example, DNA uptake via IM delivery of vaccine can be improved by the addition of sodium phosphate to the formulation. Increased DNA uptake via IM delivery can also be accomplished by electrotransfer (e.g., applying a series of electrical impulses to muscle immediately after DNA immunization). Adjuvants which can also be added to the vaccine to improve DNA stability and uptake as well as improve immune induction include water emulsions (e.g., complete and incomplete Freund's adjuvant), oil, Corynebacterium parvum, Bacillus Calmette Guerin, iron oxide, sodium alginate, aluminum hydroxide, aluminum and calcium salts (i.e., alum), unmethylated CpG motifs, glucan, and dextran sulfate. Coinjection of cytokines, ubiquitin or costimulatory molecules can also help improve immune induction. Fusions of the antigen with cytokine genes, helper epitopes, ubiquitin, or signal sequences have been successful and can also induce immune response. Fusions that aid in targeting to certain cell types can also be done. For example, antigen fused to L-selectin was successful in targeting the antigen to high endothelial venules of peripheral lymph nodes.

The medium in which the DNA vector is introduced should be physiologically acceptable for safety reasons. Suitable pharmaceutical carriers include sterile water, saline, dextrose, glucose, or other buffered solutions (e.g., TE or PBS). Included in the medium can be physiologically acceptable preservatives, stabilizers, diluents, emulsifying agents, pH buffering agents, viscosity enhancing agents, colors, etc.

Once the DNA vaccine is delivered, the nucleic acid molecules (e.g., DNA plasmids) are taken up into host cells, which then express the plasmid DNA as protein. Once expressed, the protein is processed and presented in the context of self-major histocompatibility complex (MHC) class I and class II molecules. An immune response is then generated against the DNA-encoded immunogen. To improve the effectiveness of the vaccine, multiple injections can be used for therapy or prophylaxis over extended periods of time. To improve immune induction, a prime-boost strategy can be employed. Priming vaccination with DNA and a different modality for boosting (e.g., live viral vector or protein antigen) has been used successfully in inducing cell-mediated immunity. The timing between priming and boosting varies and is adjusted for each vaccine.

Administering Nucleic Acid Compositions

The new nucleic acid compositions can be administered, or inoculated, to an individual as naked nucleic acid molecules (e.g., naked DNA plasmids) in physiologically compatible solutions such as water, saline, Tris-EDTA (TE) buffer, or in phosphate buffered saline (PBS). They can also be administered in the presence of substances (e.g., facilitating agents and adjuvants) that have the capability of promoting nucleic acid uptake or recruiting immune system cells to the site of inoculation. Nucleic acid compositions for inducing immune responses have many modes and routes of administration. They can be administered intradermally (ID), intramuscularly (IM), and by either route, they can be administered by needle injection, gene gun, or needle-less jet injection (e.g., Biojector™ (Bioject Inc., Portland, Oreg.). Other modes of administration include oral, intravenous, intraperitoneal, intrapulmonary, intravitreal, and subcutaneous inoculation. Modes of mucosal vaccination may also be employed. These include delivery, for example, via intranasal, ocular, oral, vaginal, or rectal topical routes. Delivery by these topical routes can be by nose drops, eye drops, inhalants, suppositories, or microspheres. Also possible are delivery methods which use an electroporation device, or which rely on skin surface absorption.

Suitable doses of nucleic acid compositions for humans can range from 1 μg/kg to 1 mg/kg of total nucleic acid in a composition, e.g., from 5 μg/kg-500 mg/kg of a nucleic acid composition, 10 μg/kg-250 μg/kg of a nucleic acid composition, or 10 μg/kg-170 μg/kg of a nucleic acid composition. In one embodiment, a human subject (18-50 years of age, 45-75 kg) is administered 1.2 mg-7.2 mg of a nucleic acid composition. The nucleic acid composition includes compositions containing a pool of nucleic acids encoding distinct antigens. For example, a dose of 3 mg of a nucleic acid composition encoding 3 different CMV antigens can have 1 mg of DNA encoding each antigen. DNA vaccines can be administered multiple times, e.g., between two-six times, e.g., three times. In an exemplary method, 100 μg of a DNA composition is administered to a human subject at 0, 4, and 12 weeks (100 μg per administration).

Assessing Immune Responses Induced by Nucleic Acid Compositions

Advancements in the field of immunology have allowed more thorough and sensitive evaluation of cellular responses to candidate CMV, e.g., HCMV, vaccines. Such assays as Intracellular Cytokine Staining (ICS) and ELISPOT (an enzyme-linked immunosorbent assay format), allow detecting and counting cells producing cytokines (e.g., TNFα and IFN-γ) in response to antigen. For example, isolation of splenocytes or peripheral blood monocyte cells (PBMCs) from animals or human patients followed by in vitro stimulation with HCMV epitopes such as recombinant vaccinia virus expressing pp65 or pp150, and finally testing by ELISPOT and/or intracellular cytokine staining (ICS) can determine the potential for inducing a cell-mediated immune response in a vaccine recipient. Flow cytometry using tetramers (i.e., molecules consisting of four copies of a given class I molecule bound to their cognate peptide and alkaline phosphatase) allows the enumeration of antigen-specific T cells (e.g., detection of T cells that recognize specific peptides bound to major histocompatibility complex (MHC) class I molecules). A chromium release assay allows the assessment of cytotoxicity. To assess a cell-mediated immune response to a DNA vaccine, the more traditional approaches of measuring T cell proliferation in response to antigen and CTL-mediated killing of autologous cells expressing CMV, e.g., HCMV epitopes are also available.

ELISA assays and Western blots have allowed the assessment of humoral immune responses. ELISA and Western blots can be used to assess antibody binding and antibody specificity. Neutralizing assays can determine the ability of antibody to protect against HCMV infection.

Neutralizing Antibody Responses Induced by gM and gM/gN DNA Vaccines

While the gM DNA vaccine was able to induce a neutralizing antibody response from a serum dilution of 1:64 and gN was unable to induce a detectable response due to its low expression rate, in immunized rabbits, the combination vaccine of gM and gN DNA was able to induce a much higher neutralizing antibody response (a serum dilution of 1:256) than expected. gM in combination with gN unexpectedly induced a greater than additive (synergistic) neutralizing antibody response (See Table 1, Example 2).

The combination of antisera raised by gB plus gM/gN further improved the neutralizing activity over using either antisera raised by gB alone or antisera raised by gM/N alone. (See Table 1, Example 2.)

This new information will have significant impact in the development of human CMV vaccines.

Kits

The invention also includes kits comprising the nucleic acid compositions described herein. The kits can include one or more other elements including: instructions for use; other reagents, e.g., a diluent, devices or other materials for preparing the composition for administration; pharmaceutically acceptable carriers; and devices or other materials for administration to a subject. Instructions for use can include instructions for therapeutic application (e.g., DNA vaccination and boosting) including suggested dosages and/or modes of administration, e.g., in a human subject, as described herein. Instructions can also provide directions for prophylactic treatment, e.g., in patients who are susceptible to HCMV infection, e.g., as described herein.

The kit can further contain at least one additional reagent, such as a diagnostic or therapeutic agent, e.g., a diagnostic agent to monitor a response to immune response to the compositions in the subject, or an additional therapeutic agent.

In one embodiment, the kit includes a vial (or other suitable container) containing nucleic acids encoding two, three, four, five, or six distinct CMV antigens (e.g., gcII antigens, gcIII antigens, gB, pp65, pp150, or antigenic fragments thereof). In various examples, the kit includes nucleic acids encoding one of the following combinations of CMV antigens: gcII and pp65; gcIII and pp65; gcII and pp150; gcIII and pp150; gcII, pp65, and pp150; gB, gcII, and pp65, and so on.

Administration of Vaccines to Humans

A person sera-negative for HCMV can be immunized with one or a combination of vaccines described herein. One primary objective of a CMV vaccine is to inhibit infection during pregnancy. This would imply vaccination of all sera-negative adolescent girls or older women who intend to become pregnant (e.g., greater than age 11, e.g., between the ages of 11 to 40). These include a female contemplating pregnancy or a pregnant female. Vaccination can be advised just before pregnancy is contemplated. Regular boosts can be given throughout childbearing years. Both adolescent and pediatric vaccination can be considered. Another objective of an HCMV vaccine is to induce or enhance immunity in organ (e.g., kidney, liver, heart, and lung) transplant recipients before transplant surgery or to induce or enhance immunity in HIV-infected individuals. Another objective is to induce immunity in bone marrow donors so they can serve as sources of transfused T cells active against HCMV. Another objective is to administer the vaccine to sero-positive individuals as a therapeutic application against current HCMV infection.

Formulations

Two or more immunizations may be beneficial. HCMV DNA vaccines can be delivered individually or in combination with other HCMV DNA vaccines. The multi-antigen formulation can be delivered as a premixed formulation or in multiple separate inoculations, that are administered close enough together in time that all antigens are expressed and present in the recipient at the same time.

The new HCMV DNA vaccines can be used alone or in combination with additional modalities of HCMV vaccines, e.g., live-attenuated, killed, protein or peptide based, viral or bacterial vector based can be delivered. Such a combination can be delivered sequentially (prime+boost) or concurrently in the same or more than one inoculation simultaneously. The new HCMV DNA vaccines can be used as either the priming or boosting component or both.

The HCMV gene inserts in the HCMV DNA vaccines of the invention can also be used for other forms of HCMV vaccine productions. For example, the HCMV gene inserts can be cloned into other viral or bacterial vectors. The new HCMV gene inserts can also be cloned into a recombinant protein expression system to produce HCMV proteins for vaccine and diagnostic reagents. Suitable plasmids, deliver, and dosages of nucleic acid compositions are described elsewhere herein. In various examples, suitable plasmids for administration in humans will contain promoters which permit high levels of expression of the CMV antigens (e.g., promoters that are highly active and/or constitutively active in human cells). HCMV promoters (e.g., the HCMV immediate early promoter) can be used to drive expression of the HCMV antigens.

### EXAMPLES

The invention is further described in the following examples, which do not limit the scope of the invention described in the claims.

The nucleic acid compositions described herein have the advantage of being able to raise broad immune responses, including neutralizing antibody responses and cellular immune responses against multiple CMV (e.g., HCMV) antigens. For the following examples, cDNA fragments of different CMV (e.g., HCMV) genes (e.g., gB, truncated gB (gBs), truncated gB containing a tPA leader sequence (tPA.gBs), gM, gN, gcI, gcII, gcIII, pp65, and pp150) were subcloned into an optimized mammalian expression vector, pJW4303, which uses the CMV promoter (Lu et al., Methods in Molecular Medicine, 29:355-374). Briefly, the pJW4303 vector contains, in the following order, CMV immediate early (IE) promoter (bp 1-1194), CMV IE promoter intron A (bp 1195-2027), tissue plasminogen activator (tPA) (bp2027-2102), bovine growth hormone (BGH) polyA (bp 2119-2419), pBR vector backbone (amp R) (2419-4794), and SV40 Ori (bp 4794-5139). cDNA fragments of the genes to be expressed can be inserted into the site formed by digestion with HindIII (2027) and BamHI (2119) or the site formed by digestion with NheI (2102) and BamHI (2119).

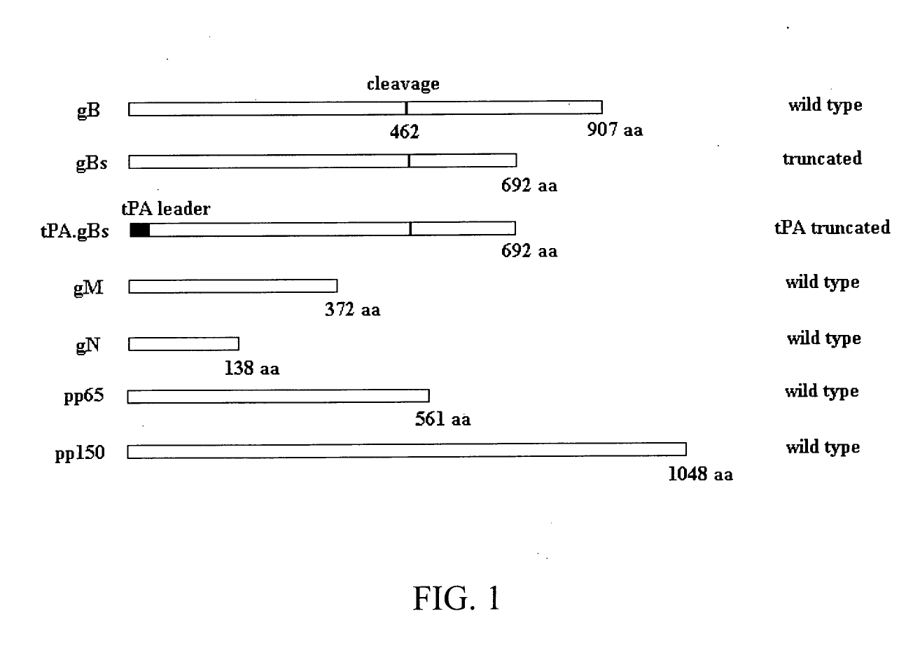

FIG. 1 shows some of the CMV genes gB, gBs, tPA.gBs, gM, gN, pp65, and pp150, which were cloned into the pJW4303 optimized vector and tested in animals (e.g., rabbits and Balb/C mice). These naked DNA plasmids were used as vaccines either individually or in combination.

### Example 1

### gB DNA Vaccine

Rabbits were immunized by gene gun with the gB DNA vaccine (6 μg) or vector control vaccine (6 μg) three times at intervals of four weeks each. Two weeks after the third immunization, blood was drawn from the rabbit and serum was isolated from the blood. Serum was then tested for ability to neutralize AD169.

Neutralization Assay

Flat bottomed 96-well microtiter plates were seeded with 2-5×10⁴ FSK cells 2-3 days prior to performing the neutralization assay. More than 80% confluence is ideal for conducting the neutralizing assay. At day 1, viral stock was incubated at 37° C. for 1 hour 0.3 ml of infectious virus (approximately 150 PFU) was mixed with 0.3 ml of diluted natural rabbit antisera (both postbleed and prebleed or negative sera). Also medium without virus was included as positive control, and 1000 pfu/well was included for positive control. After 60 minutes of incubation at 37° C., 0.2 ml of a virus-antibody mixture was added to replicate wells of a 96-well tissue culture plate containing FSK cells. Three hours later, the inoculum was removed and the monolayer was fed with fresh complete medium and incubated 16 hours.

The following day, the medium was removed and the monolayer was washed once with Dulbecco's phosphate-buffered saline, pH 7.4, and then fixed in absolute ethanol for 20 minutes at room temperature. Following fixation, the monolayer was immediately rehydrated with DPBS. The cells were than stained with 50 μl of tissue culture medium containing MAb p63-27, which is reactive against immediate-early gene 1 product pp72, for 40 minutes at 37° C. After washing the wells twice with DPBS, 50 μl of fluorescein conjugated goat anti-mouse IgG diluted 1/60 in DPBS was added and the plate incubated for 40 minutes. The wells were then washed twice with DPBS, counterstained with 0.02% Evans blue and 0.2 ml of DPBS containing 30% glycerol was added. Finally, the plate was viewed with Leitz epifluorescence microscope with a stage adapter for reading microtiter plate. Fluorescent nuclei were counted. Infectivity was expressed as fluorescent nuclei per ml of inoculum. Neutralization was expressed as the mean percent reduction in fluorescent nuclei compared with control cultures containing no antibody or control nonimmune sera. 50% reduction was used as the indicator.

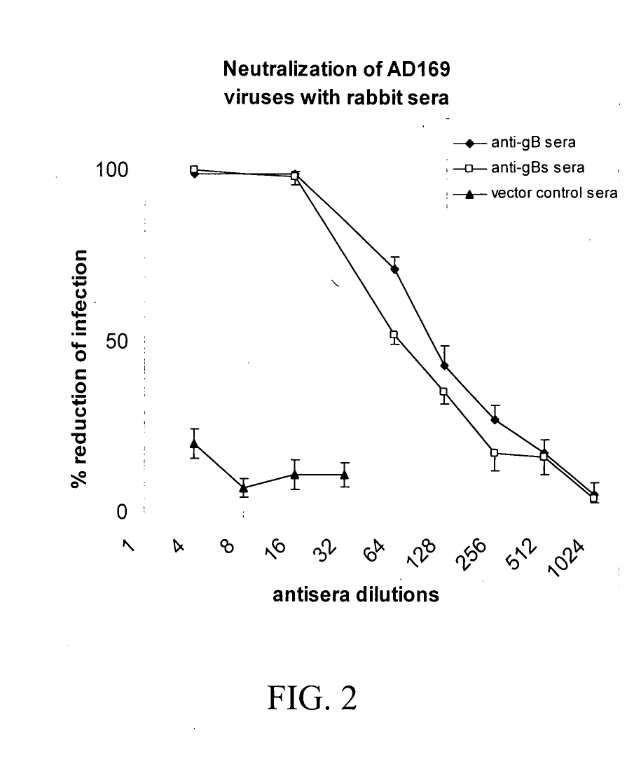

FIG. 2 is a graph showing neutralization of HCMV AD169 viruses with antisera dilutions. As seen in FIG. 2, both gB and gBs constructs induced neutralizing antibody responses, with almost 100% reduction of infection seen with antisera dilution of 1:16. Previously, gBs (which lacks the transmembrane domain) was more commonly used due to solubility issues with the full length gB (which retains the transmembrane domain). Full-length gB in the optimized pJW4303 construct was successful here as seen in FIG. 2.

### Example 2

### Co-Expression Enhances Production of gM and gN

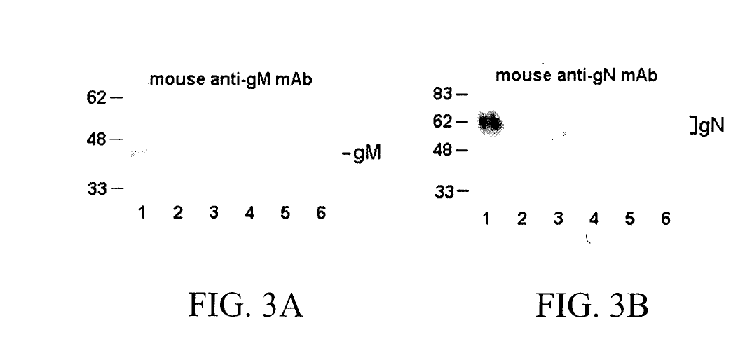

293T cells were transiently tranfected with a gM and gN-encoding DNA vaccine plasmid, a gM-encoding DNA vaccine plasmid, or a gN-encoding DNA vaccine plasmid. Lysates of transfected cells were analyzed for expression of gM and gN by western blotting. The results are depicted in FIGS. 3A and 3B. Lysates of cells transfected with gM alone (10 μg of DNA was transfected; FIG. 3A, lane 4, and FIG. 3B, lane 4) and gN alone (10 μg of DNA was transfected; FIG. 3A, lane 5, and FIG. 3B, lane 5) did not exhibit expression of gM or gN by western blotting. However, lysates of cells transfected with gM- and gN-encoding DNA did exhibit expression of each by western blotting (10 μg of each DNA was transfected; FIG. 3A, lane 3, and FIG. 3B, lane 3). Lysates of CMV-infected cells (positive control (FIG. 3A, lane 1, and FIG. 3B, lane 1) displayed expression of both gM and gN. This experiment shows that co-expression of DNA encoding both gM and gN resulted in higher levels of expression than observed when either glycoprotein was expressed alone. gM and gN, when co-expressed, form a disulfide-linked complex. Complex formation may be required for transport of the proteins from the endoplasmic reticulum to Golgi and trans-Golgi compartments (Mach et al., J. Virol., 74(24):11881-11892, 2000). Co-administration of gM- and gN-expressing nucleic acids may permit optimal expression of these gene products, leading to potent stimulation of an immune response against the antigens. Furthermore, providing these antigens via DNA vaccination eliminates the complications of preparing proteins for administration. This is particularly useful for proteins such as these, which are membrane-associated and form complexes during biosynthesis in the cell, and thus would be expensive to express, purify, and administer in protein form.

### Example 3

### gM, gN, gcII and gB+gcII DNA Vaccines

Rabbits were immunized by gene gun with the gB DNA vaccine plasmid (6 μg), the gM vaccine plasmid, the gcII (gM/N) vaccine plasmid, gB+gcII vaccine plasmid, or vector control vaccine plasmid three times at intervals of four weeks each. Two weeks after the third immunization, blood was drawn from the rabbit and serum was isolated from the blood.

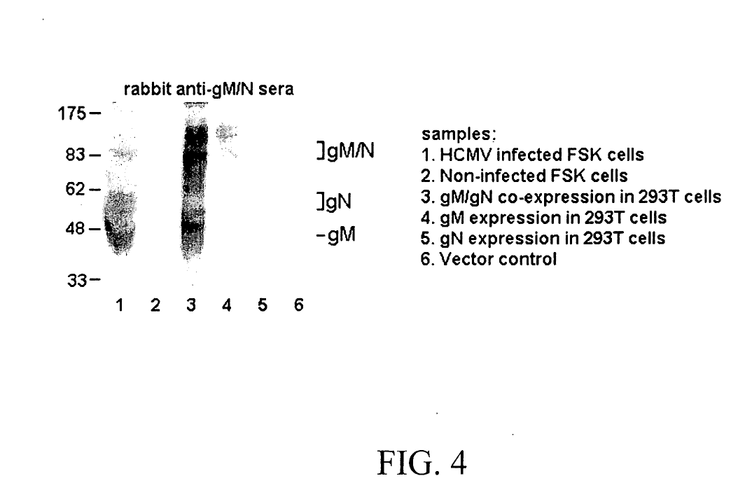

Sera were tested for recognition of gM and gN by western blotting. As shown in FIG. 4, sera from rabbits immunized with the gcII plasmid recognized protein expressed in HCMV-infected FSK cells (FIG. 4, lane 1), and gM and gN expressed in 293T cells (FIG. 4, lane 3). As noted above, the gN plasmid did not give rise to a detectable response in other tests.

Sera were also tested for neutralization of AD169. The protocol for the neutralization assay is described in Example 1, above. As seen in Table 1, while the gM vaccine alone was able to induce low titer neutralizing antibody in the immunized rabbit (and gN alone had been shown to be ineffective), the combination of gM and gN (gM/N) was able to induce a much higher, and greater than additive (synergistic), neutralizing antibody response. A higher neutralizing titer is reflected in the fact that virus was neutralized by a greater dilution of serum from gM/N-vaccinated rabbits as compared to rabbits vaccinated with gM alone. Thus, these two antigenic proteins can produce a neutralizing antibody response in the rabbit. Furthermore, the combination of gB and gM/N was able to induce a protective antibody response. These data indicate that vaccination with a combination of gB and gM/N can produce a neutralizing antibody response in rabbits that is at least additive and can therefore provide a protective response.

TABLE 1
Neutralization of AD169 (50 pfu) with DNA vaccine induced rabbit sera

| rabbit sera | 50% reduction (dilution) |
|---|---|
| Anti-gB | 1:256 |
| Anti-gM | 1:64 |
| Anti-gM/N | 1:256 |
| Anti-gB + Anti-gM/N | 1:512 |
| vector control | 1:8 |

### Example 4

### pp65 DNA Vaccine

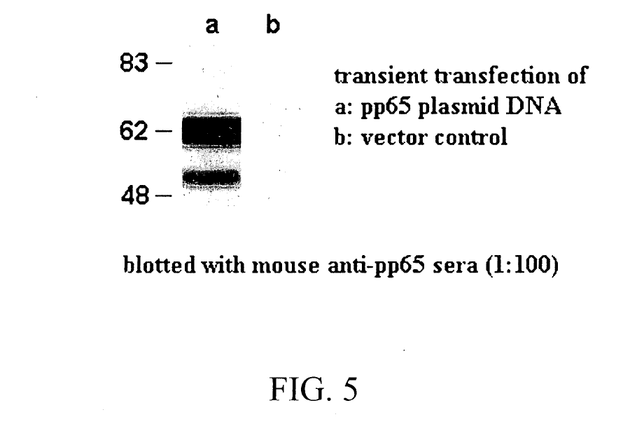

Balb/C mice were immunized by gene gun three times at four-week intervals with DNA vaccine expressing human CMV pp65. 6 μg of DNA was administered at each immunization. Two weeks after the third immunization, blood was drawn and serum was isolated from the blood and tested. As seen in FIG. 5, a 1:100 dilution of serum from mice immunized with the pp65 vaccine was able to specifically recognize antigen by western blot analysis.

At a time within 2 weeks and 6 months after the third immunization of the mice, cell-mediated immunity was tested. On the day of actual testing, the immunized mice were sacrificed, the spleen was removed and splenocytes were isolated after red blood cell lysis. Splenocytes were then tested by specific stimulation with related HCMV antigen, namely recombinant vaccinia virus expressing pp65 (VV/pp65).

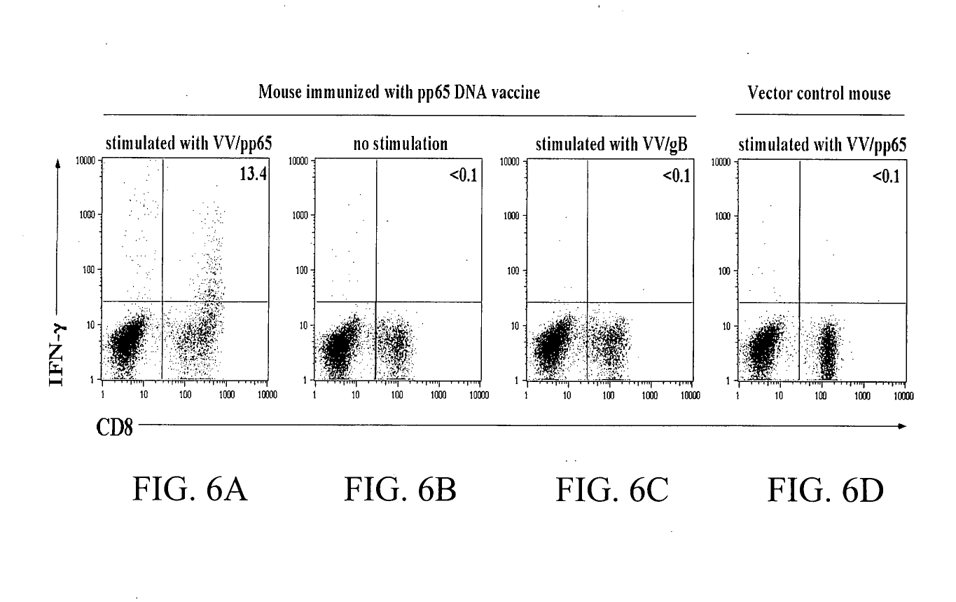

FIGS. 6A-D show the results of experiments in which mice were immunized with the pp65 vaccine. Immunized mice were capable of mounting a specific cell-mediated response to vaccinia virus VV/p65 stimulation in culture. The first three plots (FIGS. 6A-C) are results of a FACS analysis of splenocytes from a mouse immunized with pp65 vaccine. The first and fourth plots show splenocyte response to stimulation with vaccinia virus VV/pp65 in culture, the fourth plot (FIG. 6D) being the negative control of splenocytes from a mouse immunized with vector control alone. The second plot serves as a negative control in which the splenocytes were not stimulated. The third plot is another negative control of splenocytes from a pp65 vaccinated mouse; the splenocytes were stimulated with recombinant vaccinia virus VV/gB expressing unrelated antigen gB in culture. Absence of response in the third plot signifies a pp65-specific response in the first plot.

In FIGS. 6A-D, the x-axis represents presence of the cell surface marker CD8 on the surface of splenocytes, meaning that a dot to the right of the verticle line represents a splenocyte positive for CD8 on its surface (CD8 positive or CD8+), while a dot to the left of the verticle line represents a splenocyte that does not have CD8 on its surface (CD8 negative or CD8−). The y-axis represents presence of IFN-γ inside T cells, meaning that a dot above the horizontal line represents a functional splenocyte producing IFN-γ, while a dot below the horizontal line represents a splenocyte not producing IFN-γ. The upper right quadrant represents splenocytes positive for both CD8 and IFN-γ staining, which are considered antigen-specific CD8+ lymphocytes.

In FIGS. 6A-D, the numbers in the upper right comer of each plot give the ratio of dots occurring in the upper right quadrant relative to the total dots in the right half of the plot. These double positive cells represent splenocytes that mount a specific CD8 T cell response to the stimulation of related antigen pp65 expressed by the recombinant vaccinia virus VV/pp65 as they are not present in the unstimulated or stimulated with unrelated antigen plots. The presence of cells in the upper right quadrant in this first plot shows that the splenocytes from a mouse vaccinated with the pp65 vaccine were able to produce a pp65-specific response. The absence of the representation of cells in the upper right quadrant in the third plot shows that a splenocyte response was not detectably produced in response to stimulation of unrelated antigen gB expressed by recombinant vaccinia virus VV/gB, showing that vaccination with the pp65 DNA vaccine can induce splenocytes that can specifically respond to pp65 stimulation, but not to gB stimulation.

The percentages of pp65-specific CD8⁺ T cells in splenocytes from five individual mice immunized with the pp65 vaccine are listed in Table 2. These percentages were determined by FACS staining of cells and reflect percentages of CD8⁺ cells which stained positive for IFN-γ in response to stimulation with pp65 or non-specific (“unrelated”) antigens. As shown in Table 2, an average of 3.49 percent of total splenocytes from pp65-immunized mice were pp65-specfic CD8⁺ T cells. Percentages in individual mice ranged from 1.3 to 13.4 percent. In all mice, less than 0.1 percent of cells were specific for the unrelated antigen.

TABLE 2
CMV pp65 specific CD8⁺ T cell responses from mice immunized with
pp65 DNA vaccine (percentage of CD8⁺ T cells stained positive
for IFN-γ secretion)

| stimulation | 1 | 2 | 3 | 4 | 5 | avg |
|---|---|---|---|---|---|---|
| pp65 | 1.49 | 4.13 | 1.3 | 4.83 | 13.4 | 3.49 |
| unrelated | <0.1 | <0.1 | <0.1 | <0.1 | <0.1 | <0.1 |

CD4⁺ T cell responses in individual mice were also determined. Table 3 lists the percentages of CD4⁺, pp65-specific (i.e., IFN-γ-secreting) splenocytes as determined by FACS. An average of 0.71 percent of splenocytes from immunized mice were pp65-specific CD4⁺ T cells. Percentages in individual mice ranged from 0.31 to 3.99 percent. In all mice, less than 0.1 percent of cells were specific for the unrelated antigen.

TABLE 3
CMV pp65 specific CD4+ T cell responses from mice immunized with
pp65 DNA vaccine (percentage of CD4+ T cells stained positive
for IFN-γ secretion).

| | | | | | | |
|---|---|---|---|---|---|---|
| mouse | 1 | 2 | 3 | 4 | 5 | avg |

| | | | | | | |
|---|---|---|---|---|---|---|
| pp65 | 0.31 | 0.59 | 0.32 | 0.78 | 3.99 | 0.71 |
| unrelated | <0.1 | <0.1 | <0.1 | <0.1 | <0.1 | <0.1 |

These data show that the pp65 vaccine induced a robust antigen-specific CD8⁺ and CD4⁺ T cell-mediated immune response in mice.

### Example 5

### pp150 Vaccine

Balb/C mice were immunized by gene gun three times at four-week intervals with DNA vaccine expressing human CMV pp150. 6 μg of DNA was administered at each immunization.

At a point between 2 weeks and 6 months after the third immunization of the mice, cell-mediated immunity was tested. On the day of actual testing, the immunized mice were sacrificed, the spleen was removed, and splenocytes were isolated after red blood cell lysis. Splenocytes were then tested by specific stimulation with related HCMV antigen, namely recombinant vaccinia virus expressing pp150 (VV/pp150).

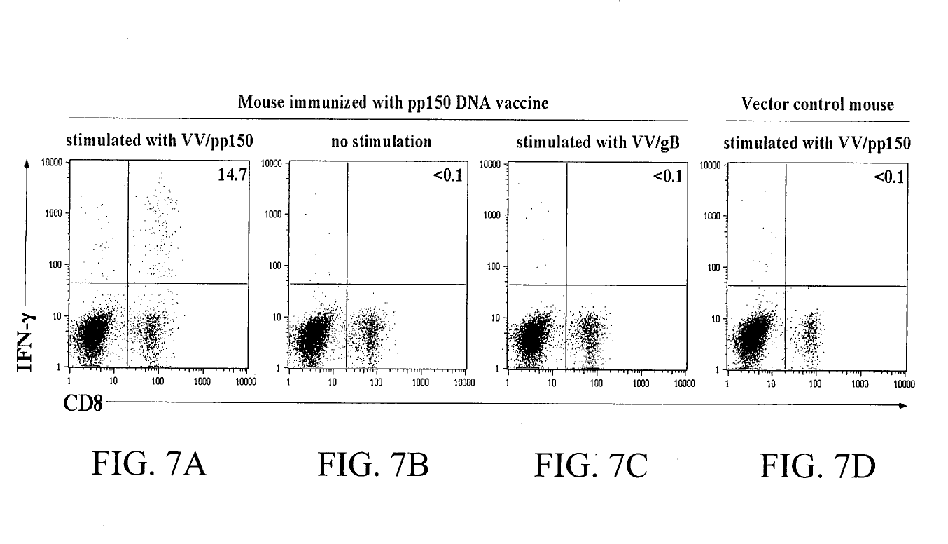

FIGS. 7A-D show the results of experiments in which mice were immunized with the pp150 vaccine. The results indicate that immunized mice were capable of mounting a specific cell-mediated response to vaccinia virus VV/pp150 stimulation. The first three plots (FIGS. 7A-C) are results of a FACS analysis of splenocytes from a mouse immunized with the pp150 DNA vaccine. The first and fourth plots show splenocyte response to stimulation with vaccinia virus VV/pp150, the fourth plot (FIG. 7D) being the negative control. The second plot serves as a negative control in which the splenocytes were not stimulated. The third plot is another negative control of splenocytes from a pp150 vaccinated mouse. The splenocytes in the third plot were stimulated with recombinant vaccinia virus VV/gB expressing unrelated antigen gB. Absence of response in the third plot signifies a pp150-specific response in the first plot.

The x-axis of FIGS. 7A-D represents the presence of the cell surface marker CD8 on the surface of splenocytes, meaning that a dot to the right of the verticle line represents a splenocyte positive for CD8 on its surface (CD8 positive or CD8+) while a dot to the left of the verticle line represents a cell that does not have CD8 on its surface (CD8 negative or CD8−). The y-axis represents the presence of IFN-γ inside T cells, meaning that a dot above the horizontal line represents a functional splenocyte producing IFN-γ while a dot below the horizontal line represents a splenocytes not producing IFN-γ. Dots in the upper right hand quadrant represent splenocytes positive for both CD8 and IFN-γ staining, which are considered as antigen-specific CD8+ lymphocytes.

In FIGS. 7A-D, the numbers in the upper right corner of each plot give the ratio of dots occurring in the upper right quadrant relative to the total dots in the right half of the plot. These double positive cells represent splenocytes that mount a specific CD8 T cell response to the stimulation of related antigen expressed by the recombinant vaccinia virus VV/pp150 as they are not present in the unstimulated or stimulated with unrelated antigen plots. The presence of cells in the upper right quadrant in this first-plot shows that the pp150 vaccine was able to induce splenocytes that could respond specifically to a pp150 stimulation. The absence of representation of cells in the upper right quadrant in the third plot shows that a splenocyte response was not detectably produced in response to stimulation of unrelated antigen gB expressed by recombinant vaccinia virus VV/gB, showing that vaccination with the pp150 DNA vaccine can induce splenocytes that respond specifically to pp150 stimulation, but not to gB stimulation.

The percentages of pp150-specific CD8⁺ T cells in splenocytes from 5 individual mice immunized with the pp150 vaccine are listed in Table 4. These percentages were determined by FACS staining of cells and reflect percentages of CD8⁺ cells which stained positive for IFN-γ in response to stimulation with pp150 or non-specific (“unrelated”) antigens. As shown, an average of 5.12 percent of total splenocytes from pp150-immunized mice were pp150-specfic CD8⁺ T cells, with percentages in individual mice ranging from 1.05 to 13.4 percent. In all mice, less than 0.1 percent of cells were specific for the unrelated antigen.

TABLE 4
CMV pp150 specific CD8⁺ T cell responses from mice immunized
with pp150 DNA vaccine

| stimulation | 1 | 2 | 3 | 4 | 5 | avg |
|---|---|---|---|---|---|---|
| pp150 | 4.16 | 3.68 | 1.05 | 16.3 | 13.4 | 5.12 |
| Unrelated antigen | <0.1 | <0.1 | <0.1 | <0.1 | <0.1 | <0.1 |

These data show that the pp150 vaccine induced a robust antigen-specific CD8⁺ T cell mediated response in mice.

### Example 6

### gH, gL, gO, and gcIII Vaccines

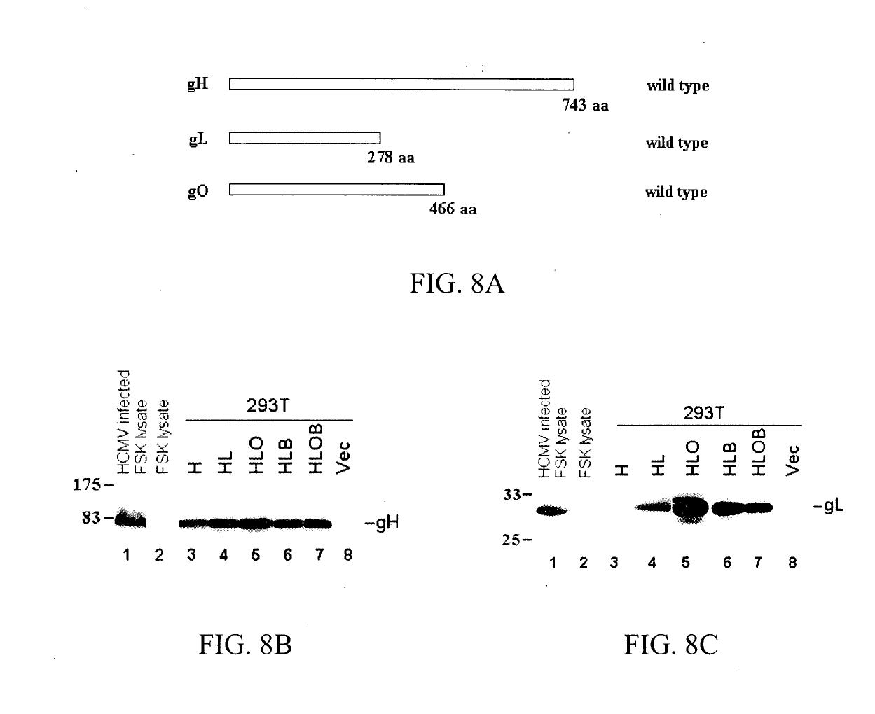

CMV gH, gL, and gO genes were cloned into the pJW4303 optimized vaccine vector. The gH, gL, and gO polypeptides are depicted schematically in FIG. 8A. gH is the wild type glycoprotein H, which is 743 amino acids long. gL is the wild type glycoprotein L, which is 278 amino acids in length. gO is the wild type glycoprotein O, which is 466 amino acids in length.

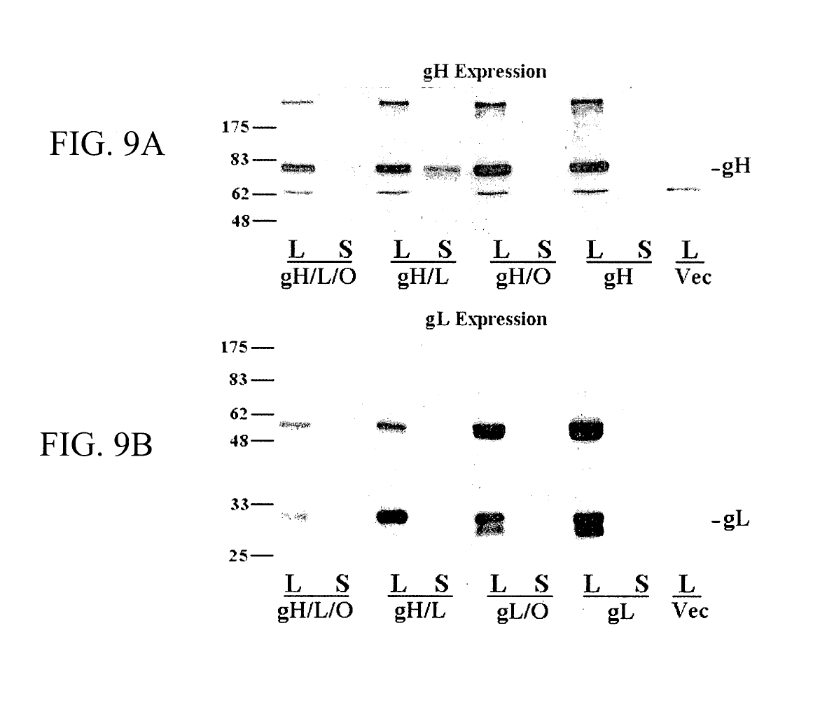

DNA vaccine vectors encoding each of these were transiently transfected into 293T cells in the following combinations: gH alone, gH+gL, gH+gL+gO, gH+gL+gB, gH+gL+gO+gB. Lysates of cell transfectants were analyzed for expression of gH and gL by western blotting. As shown in FIG. 8B, all of the transfectants into which a gH-expressing vector was introduced exhibited expression of gH. As shown in FIG. 8C, all of the transfectants into which a gL-expressing vector was introduced exhibited expression of gL. Positive control samples (i.e., CMV-infected cells, lane 1 of FIG. 8B and FIG. 8C) showed expression of gH and gL. Negative control samples (uninfected cells, lane 2 of FIG. 8B and FIG. 8C; and 293T cell transfected with empty vector, lane 8 of FIG. 8B and FIG. 8C) did not exhibit expression of gH or gL. These data show that gH and gL can be expressed from DNA vaccine vectors in mammalian cells. Previous studies suggested that co-expression of gL facilitated proper processing and trafficking of gH through the secretory pathway (Spaete et al., Virology, 193(2):853-861, 1993). We observed expression of each, without dependence on co-expression of the other, in contrast to our observation with gM and gN. However, we analyzed secreted and cell-associated forms of each protein by western blot. Cell lysate (“L”) and cell supernatants (“S”) of gH/L/O, gH/gL, gH/O, gH, and empty vector transfectants were resolved by SDS-PAGE and blotted for gH and gL expression. As shown in FIG. 9, upper panel, co-expression of gH and gL allows secretion of a form of gH, which was recovered from cell supernatants (FIG. 9, upper panel, compare lane 4 to lanes 6 and 8). These data indicate that co-administration of gH and gL DNA vaccines is not required for efficient expression of each gene product. However, co-expression may facilitate the secretion of gH.

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A composition comprising a plurality of sets of nucleic acid molecules, each set of nucleic acid molecules encoding a different type of cytomegalovirus (CMV) polypeptide, and each molecule of a set encoding the same type of CMV polypeptide, wherein one or more sets of the plurality encodes a CMV polypeptide that induces a neutralizing antibody response, and one or more sets of the plurality encodes a CMV polypeptide that induces a cell-mediated immune response.

2. The composition of claim 1, wherein the nucleic acid molecules comprise DNA plasmids.

3. The composition of claim 1, wherein the CMV polypeptides are human CMV (HCMV) polypeptides.

4. The composition of claim 1, wherein the CMV polypeptides that induce an antibody response are selected from the group consisting of glycoprotein B, glycoprotein complex II, and glycoprotein complex III, and antigenic fragments thereof.

5. The composition of claim 1, wherein the CMV polypeptides that induce an antibody response comprise glycoprotein B and glycoprotein complex II, or antigenic fragments thereof.

6. The composition of claim 1, wherein the CMV polypeptides that induce a cell-mediated immune response are selected from the group consisting of phosphoprotein pp65 (pp65), phosphoprotein pp150 (pp150), and antigenic fragments thereof.

7. A composition comprising a plurality of sets of nucleic acid molecules, each set encoding a different type of human cytomegalovirus (HCMV) polypeptide that induces a neutralizing antibody response, and each nucleic acid molecule of a set encoding the same type of HCMV polypeptide.

8. The composition of claim 7, wherein the CMV polypeptides that induce an antibody response consist of glycoprotein B and glycoprotein complex II, or antigenic fragments thereof.

9. A composition comprising a plurality of sets of nucleic acid molecules, each set encoding a different type of human cytomegalovirus (HCMV) polypeptide that induces a cell-mediated immune response, and each nucleic acid molecule of a set encoding the same type of HCMV polypeptide.

10. The composition of claim 9, wherein the polypeptides that induce a cell-mediated response comprises pp65 and pp150.

11. The composition of claim 7, wherein the polypeptides that induce a neutralizing antibody response are selected from the group consisting of glycoprotein B, gM, gN, a combination of gM and gN (glycoprotein complex II; gcII), and a combination of gH, gL, and gO (glycoprotein complex III; gcIII) of HCMV, and antigenic fragments thereof.

12. The composition of claim 9, wherein the polypeptides that induce a cell-mediated immunity response comprise phosphoprotein 65, phosphoprotein 150, both phosphoprotein 65 and phosphoprotein 150, or antigenic fragments thereof.

13. The composition of claim 7, wherein the polypeptides that induce a neutralizing antibody response comprise gcII or antigenic fragments thereof.

14. The composition of claim 7, wherein the polypeptides that induce a neutralizing antibody response comprise gcIII or antigenic fragments thereof.

15. The composition of claim 7, wherein the polypeptides that induce a neutralizing antibody response comprise gB and gcII or antigenic fragments thereof.

16. The composition of claim 7, wherein the polypeptides that induce a neutralizing antibody response consist of gB and gcIII or antigenic fragments thereof.

17. A pharmaceutical composition that elicits an immune response against human cytomegalovirus (HCMV) comprising the composition of claim 1 and a pharmaceutically acceptable carrier.

18. A pharmaceutical composition that elicits an immune response against human cytomegalovirus (HCMV) comprising the composition of claim 7 and a pharmaceutically acceptable carrier.

19. A pharmaceutical composition that elicits an immune response against human cytomegalovirus (HCMV) comprising the composition of claim 9 and a pharmaceutically acceptable carrier.

20. A method of eliciting an immune response against human cytomegalovirus (HCMV) in a subject, the method comprising administering to the subject an amount of a pharmaceutical composition of claim 17 effective to elicit an immune response against HCMV in the subject.

21. The method of claim 20, wherein administration is by needle injection, needle-less jet injection, gene gun, topical administration, surgical administration, or mucosal administration.

22. The method of claim 20, wherein the subject is a non-human mammal or a human.

23. The method of claim 22, wherein the human is sero-negative for HCMV.

24. The method of claim 23, wherein the sero-negative human is selected from the group consisting of a female between the ages of eleven and forty, a female contemplating pregnancy, a pregnant female, an HIV-infected individual, a future organ transplant recipient, and a future bone marrow donor.

25. The method of claim 22, wherein the human is sero-positive for HCMV.

26. A kit comprising the composition of claim 1 and instructions for administration of the composition to a subject in an amount effective to treat a CMV infection.

27. The kit of claim 26, wherein the amount is effective to inhibit a future CMV infection.

28. The kit of claim 26, wherein the amount is effective to treat an existing CMV infection.

29. The kit of claim 26, wherein the composition comprises DNA plasmids.

30. A kit comprising the composition of claim 7 and instructions for administration of the composition to a subject in an amount effective to treat a CMV infection.

31. A kit comprising the composition of claim 9 and instructions for administration of the composition to a subject in an amount effective to treat a CMV infection.


SEQUENCE LISTING (16 sequences)

SEQ ID NO: 1 - DNA, 2721 bp, Human cytomegalovirus
  CDS (1)...(2718)
atggaatccaggatctggtgcctggtagtctgcgttaacctgtgtatcgtctgtctgggtgctgcggtttcctcttctagtacttcccatgcaacttcttctactcacaatggaagccatacttctcgtacgacgtctgctcaaacccggtcagtctattctcaacacgtaacgtcttctgaagccgtcagtcatagagccaacgagactatctacaacactaccctcaagtacggagatgtggtgggagtcaacactaccaagtacccctatcgcgtgtgttctatggcccagggtacggatcttattcgctttgaacgtaatatcatctgcacctcgatgaagcctatcaatgaagacttggatgagggcatcatggtggtctacaagcgcaacatcgtggcgcacacctttaaggtacgggtctaccaaaaggttttgacgtttcgtcgtagctacgcttacatctacaccacttatctgctgggcagcaatacggaatacgtggcgcctcctatgtgggagattcatcacatcaacaagtttgctcaatgctacagttcctacagccgcgttataggaggcacggttttcgtggcatatcatagggacagttatgaaaacaaaaccatgcaattaattcccgacgattattccaacacccacagtacccgttacgtgacggtcaaggatcagtggcacagccgcggcagcacctggctctatcgtgagacctgtaatctgaactgtatgctgaccatcactactgcgcgctccaagtatccttatcatttttttgcaacttccacgggtgatgtggtttacatttctcctttctacaacggaaccaatcgcaatgccagctactttggagaaaacgccgacaagtttttcattttcccgaactacaccatcgtttccgactttggaagacccaacgctgcgccagaaacccataggttggtggcttttctcgaacgtgccgactcggtgatctcttgggatatacaggacgagaagaatgtcacctgccagctcaccttctgggaagcctcggaacgtactatccgttccgaagccgaagactcgtaccacttttcttctgccaaaatgactgcaacttttctgtctaagaaacaagaagtgaacatgtccgactccgcgctggactgcgtacgtgatgaggctataaataagttacagcagattttcaatacttcatacaatcaaacatatgaaaaatacggaaacgtgtccgtcttcgaaaccagcggcggtctggtggtgttctggcaaggcatcaagcaaaaatctttggtggaattggaacgtttggccaatcgatccagtctgaatatcactcataggaccagaagaagtacgagtgacaataatacaactcatttgtccagcatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggaatcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaagtacgtggaggacaaggtagtcgacccgctaccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagccttcaccatcatcctcgtggccatagccgtagtcattatcacttatttgatctatactcgacagcggcgtctgtgcacgcagccgctgcagaacctctttccctatctggtgtccgccgacgggaccaccgtgacgtcgggcagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcagaacggtacagattctttggacggacagactggcacgcaggacaagggacagaagcctaacctgctagaccggctgcgacatcgcaaaaacggctacagacacttgaaagactccgacgaagaagagaacgtctga

SEQ ID NO: 2 - protein, 906 aa, Human cytomegalovirus
MESRIWCLVVCVNLCIVCLGAAVSSSSTSHATSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHRTRRSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGQTGTQDKGQKPNLLDRLRHRKNGYRHLKDSDEEENV

SEQ ID NO: 3 - DNA, 1119 bp, Human cytomegalovirus
  CDS (1)...(1116)
atggccccctcgcacgtggataaggtgaatacacggacatggagcgcttctatcgttttcatggtgctgacttttgtcaacgtcagcgtgcatctagtgctgagcaattttccgcacctgggctacccctgcgtctactatcacgtcgtggactttgaaaggctcaacatgtcggcctacaacgtaatgcacctgcacacgcctatgcttttcttagactcggtgcagttggtgtgctacgccgtgttcatgcagctcgtctttttagccgtgaccatctactacctggtatgctggatcaagatcagcatgcgcaaggacaaaggcatgagcctaaaccagtcgacacgcgacatttcgtacatgggcgacagcctcacagccttcctcttcattctcagcatggacacgttccaactattcacactgaccatgtcatttcggctgcccagcatgatcgccttcatggccgccgtgcactttttctgcctgaccattttcaacgtgagcatggtcacgcagtaccgcagctacaaacgctcactctttttcttctcgcgtctgcaccccaagctcaaaggtacggtgcagttccgcacgctcatcgtcaacctggtagaggtagcgcttggtttcaacaccaccgtggtagccatggccctgtgctacggcttcggaaacaactttttcgtgcgtacaggccacatggtgttagccgtcttcgtggtctacgctatcatctccatcatctactttttactgatcgaggccgtcttttttcaatacgtcaaggtgcaattcggctaccacctgggcgccttctttggactctgcggcctcatctaccccatcgtgcagtacgataccttcctcagcaacgaataccgcaccggcatcagctggtcgttcggcatgctctttttcatatgggccatgtttacgacgtgtcgcgccgtccgctactttcgcggacgcggtagcggcagtgtcaagtaccaggcgctggccacagcctccggcgaagaagtcgctgtgctcagtcaccacgacagcttggaaagccgtcgcctccgcgaagaagaggacgacgacgatgatgaagacttcgaggacgcttaa

SEQ ID NO: 4 - protein, 372 aa, Human cytomegalovirus
MAPSHVDKVNTRTWSASIVFMVLTFVNVSVHLVLSNFPHLGYPCVYYHVVDFERLNMSAYNVMHLHTPMLFLDSVQLVCYAVFMQLVFLAVTIYYLVCWIKISMRKDKGMSLNQSTRDISYMGDSLTAFLFILSMDTFQLFTLTMSFRLPSMIAFMAAVHFFCLTIFNVSMVTQYRSYKRSLFFFSRLHPKLKGTVQFRTLIVNLVEVALGFNTTVVAMALCYGFGNNFFVRTGHMVLAVFVVYAIISIIYFLLIEAVFFQYVKVQFGYHLGAFFGLCGLIYPIVQYDTFLSNEYRTGISWSFGMLFFIWAMFTTCRAVRYFRGRGSGSVKYQALATASGEEVAVLSHHDSLESRRLREEEDDDDDEDFEDA

SEQ ID NO: 5 - DNA, 417 bp, Human cytomegalovirus
  CDS (1)...(414)
atggagtggaacacactagtattaggtcttttagttttatcggtagtggcagagagttctggtaacaattcatccacgtcaacctctgcaactacatcaaagtcttctgctagcgtatcaactaccaaactaacaacagttgcaacaacttctgcaacaactacgacgactacgaccttatcgacaactagcactaaactcagttctaccacccacgatcctaatgtgatgagacgacatgcgaacgatgatttttacaaggcgcattgcacatcgcatatgtatgagctctcactgtccagctttgcggcctggtggactatgcttaatgctctaattctcatgggagctttttgtattgtactacgacattgctgcttccagaactttactgcaaccaccaccaaaggctattga

SEQ ID NO: 6 - protein, 138 aa, Human cytomegalovirus
MEWNTLVLGLLVLSVVAESSGNNSSTSTSATTSKSSASVSTTKLTTVATTSATTTTTTTLSTTSTKLSSTTHDPNVMRRHANDDFYKAHCTSHMYELSLSSFAAWWTMLNALILMGAFCIVLRHCCFQNFTATTTKGY

SEQ ID NO: 7 - DNA, 2232 bp, Human cytomegalovirus
  CDS (1)...(2229)
atgcggcccggcctccccccctacctcactgtcttcaccgtctacctcctcagtcacctaccttcgcaacgatatggcgcggacgccgcatccgaagcgctggaccctcacgcatttcacctactactcaacacctacgggagacccatccgcttcctgcgtgaaaacaccacccagtgcacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctagaaagataccaacagagacttaacacctacgcattggtatccaaagacctggccagctaccgatctttttcgcagcagctgaaggcacaagacagcctgggtcagcagcccaccaccgtgccaccgcccattgatctgtcaatacctcacgtttggatgccaccccaaaccactccacacgactggaagggatcgcacaccacctcgggactacatcggccacactttaaccagacctgtatcctctttgatggacacgatctgcttttcagcaccgttacgccctgtctgcaccagggcttttacctcatggacgaactacgttacgttaaaatcacactgaccgaggacttcttcgtagttacggtatctatagacgacgacacacccatgctgcttatcttcggtcatcttccacgcgtactcttcaaagcgccctatcaacgcgacaactttatactacgacaaactgaaaaacacgagctcctggtactagttaagaaagctcaactaaaccgtcactcctatctcaaagactcggactttctcgacgccgcactcgacttcaactacctggacctcagcgcactgttacgtaacagctttcaccgttacgctgtagacgtactcaaaagcggtcgatgtcaaatgttggaccgccgcacggtagaaatggccttcgcctacgcattagcactgttcgcggcagcccgacaagaagaggccggcaccgaaatctccatcccacgagccctagaccgccaggccgcactcttacaaatacaagaatttatgatcacctgcctctcacaaacaccaccacgcaccacattgctgctatatcccacagccgtggacctggccaaacgagccctctggacgccggaccagatcaccgacatcaccagcctcgtacgcctggtctacatactttctaaacagaatcagcaacatctcattccccagtgggcactacgacagatcgccgactttgccctacaattacacaaaacgcacctggcctcttttctttcagccttcgcgcgccaagaactctacctcatgggcagcctcgtccactccatgttggtacatacgacggagagacgcgaaatcttcatcgtagaaacgggcctctgttcattggccgagctatcacactttacgcagttgctagctcatccgcaccacgaatacctcagcgacctgtacacaccctgttccagtagcgggcgacgcgatcactcgctcgaacgcctcacgcgtctcttccccgatgccaccgttcctgctaccgttcccgccgccctctccatcctatctaccatgcaaccaagcacgctggaaaccttccccgacctgttttgtctgccgctcggcgaatccttctccgcgctaaccgtctccgaacacgtcagttatgtcgtaacaaaccagtacctgatcaaaggtatctcctaccctgtctccaccaccgtcgtaggccagagcctcatcatcacccaaacggacagtcaaactaaatgcgaactaacgcgcaacatgcacaccacacacagcatcacagcggcgctcaacatttcactagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtcctagaagtaactgacgtcgtcgtggacgccaccgacagtcgtctcctcatgatgtccgtctacgcgctatcggccatcatcggcatctatctgctctaccgcatgctcaagacatgctga

SEQ ID NO: 8 - protein, 743 aa, Human cytomegalovirus
MRPGLPPYLTVFTVYLLSHLPSQRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKAQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQTKCELTRNMHTTHSITAALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 9 - DNA, 921 bp, Human cytomegalovirus
  CDS (1)...(918)
cgttttagggatcgaagacctgagcgccaactttcggcgccaactggctccttaccgtcacactctcatcgtgccgcagacttgatgtgccgccgcccggattgcggcttctctttctcacctggaccggtggtactgctgtggtgttgccttctgctgcccattgtttcctcagtcgccgtcagcgtcgctcctaccgccgccgagaaagtccccgcggagtgccccgaactaacgcgtcgatgcctgttgggtgaggtgtttcagggtgacaagtatgaaagttggctgcgcccgttggtgaatgttaccagacgcgatggcccgctatcgcaacttattcgttaccgtcccgttacgccggaggccgccaactccgtgctgttggacgatgctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacactcttttacggcctgtacaacgcagtgaaggaattctgcctgcgtcaccagctggacccgccgctactacgccacctagataaatactacgccggactgccgcccgagctgaagcagacgcgcgtcaacctgccggctcactcgcgctatggccctcaagcagtggatgctcgctaa

SEQ ID NO: 10 - protein, 306 aa, Human cytomegalovirus
RFRDRRPERQLSAPTGSLPSHSHRAADLMCRRPDCGFSFSPGPVVLLWCCLLLPIVSSVAVSVAPTAAEKVPAECPELTRRCLLGEVFQGDKYESWLRPLVNVTRRDGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 11 - DNA, 1401 bp, Human cytomegalovirus
  CDS (1)...(1398)
atggggagaaaagagatgatggtgagagacgtccctaagatggtgtttctaatatctatatctttcttgcttgtttctttcataaactgtaaagttatgtcaaaagcgctttataatcgtccttggaggggcttggtactgtctaagataggcaaatataaattagatcagcttaagttagaaattttgagacaactagaaacgactatttctacaaaatacaatgtaagtaaacaaccggttaaaaatctcactatgaacatgacagagtttccacaatactacattttagcgggccccattcagaattatagtataacctatctgtggtttgatttttatagtacccagcttagaaaacccgcaaaatacgtttactcacagtacaatcatacggctaaaacgataacattcagacccccaccttgtggtactgtgccttccatgacttgtctttccgaaatgctaaacgtttccaaacgtaatgatactggcgaacaaggttgcggtaatttcaccacgttcaaccccatgtttttcaatgtaccgcgttggaacaccaaattgtacgtgggtccgactaaggttaacgtagatagtcaaacgatttattttctaggtttaaccgccctgcttttacgttacgcacaacgcaactgtacacacagtttctacctggttaacgccatgagccggaatctatttcgcgtccccaagtatattaacggcaccaagttaaaaaacactatgcgaaaactaaaacgtaaacaagcgcccgttaaggaacaattcgaaaaaaaagctaagaaaactcagagtactactacgccatacttttcctatacaacgtctgccgctctcaacgtcactactaacgtgacttatagtattactaccgccgcaaggcgggtttccacgtctacaattgcttatcgtcctgatagcagctttatgaagtccattatggccacacagttaagggacctagcaacgtgggtgtataccactctacgttaccggcaaaatcctttttgtgaaccaagccgcaaccgaaccgccgtgtcagaatttatgaaaaacacgcacgtactaatccgtaacgaaacgccgtacactatttacggtactctcgacatgagctccttatattacaacgaaaccatgttcgtggaaaacaaaacagcttccgatagtaacaaaactacacctacgtcaccatcaatggggtttcagagaacatttatagatcccctgtgggactatctagactcgctgctgtttctagatgagattcgtaactttagcctccggtcacccacgtatgtaaaccttaccccgccggaacaccgccgggctgtaaatctgtccaccctcaatagcctttggtggtggttgcagtaa

SEQ ID NO: 12 - protein, 466 aa, Human cytomegalovirus
MGRKEMMVRDVPKMVFLISISFLLVSFINCKVMSKALYNRPWRGLVLSKIGKYKLDQLKLEILRQLETTISTKYNVSKQPVKNLTMNMTEFPQYYILAGPIQNYSITYLWFDFYSTQLRKPAKYVYSQYNHTAKTITFRPPPCGTVPSMTCLSEMLNVSKRNDTGEQGCGNFTTFNPMFFNVPRWNTKLYVGPTKVNVDSQTIYFLGLTALLLRYAQRNCTHSFYLVNAMSRNLFRVPKYINGTKLKNTMRKLKRKQAPVKEQFEKKAKKTQSTTTPYFSYTTSAALNVTTNVTYSITTAARRVSTSTIAYRPDSSFMKSIMATQLRDLATWVYTTLRYRQNPFCEPSRNRTAVSEFMKNTHVLIRNETPYTIYGTLDMSSLYYNETMFVENKTASDSNKTTPTSPSMGFQRTFIDPLWDYLDSLLFLDEIRNFSLRSPTYVNLTPPEHRRAVNLSTLNSLWWWLQ

SEQ ID NO: 13 - DNA, 1686 bp, Human cytomegalovirus
  CDS (1)...(1683)
atggagtcgcgcggtcgccgttgtcccgaaatgatatccgtactgggtcccatttcggggcacgtgctgaaagccgtgtttagtcgcggcgatacgccggtgctgccgcacgagacgcgactcctgcagacgggtatccacgtacgcgtgagccagccctcgctgatcttggtatcgcagtacacgcccgactcgacgccatgccaccgcggcgacaatcagctgcaggtgcagcacacgtactttacgggcagcgaggtggagaacgtgtcggtcaacgtgcacaaccccacgggccgaagcatctgccccagccaggagcccatgtcgatctatgtgtacgcgctgccgctcaagatgctgaacatccccagcatcaacgtgcaccactacccgtcggcggccgagcgcaaacaccgacacctgcccgtagctgacgctgtgattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctgacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctgttgatgaacgggcagcagatcttcctggaggtacaagccatacgcgagaccgtggaactgcgtcagtacgatcccgtggctgcgctcttctttttcgatatcgacttgctgctgcagcgcgggcctcagtacagcgagcaccccaccttcaccagccagtatcgcatccagggcaagcttgagtaccgacacacctgggaccggcacgacgagggtgccgcccagggcgacgacgacgtctggaccagcggatcggactccgacgaagaactcgtaaccaccgagcgcaagacgccccgcgtcaccggcggcggcgccatggcgggcgcctccacttccgcgggccgcaaacgcaaatcagcatcctcggcgacggcgtgcacgtcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggaattcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggttga

SEQ ID NO: 14 - protein, 561 aa, Human cytomegalovirus
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTSGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 15 - DNA, 3147 bp, Human cytomegalovirus
  CDS (1)...(3144)
atgagtttgcagtttatcggtctacagcggcgcgatgtggtagccctggtcaactttctgcgccatctcacgcaaaagcccgacgtggatctcgaggcacaccccaagatcctgaaaaaatgtggcgaaaaacgcctgcaccggcgtacggtgctgttcaacgagctcatgctttggttgggatactaccgcgagctgcgttttcacaaccccgacctctcctcagtgctcgaggagttcgaggtgcgttgcgtggccgtggcgcgtcgcggctacacttacccgttcggtgatcgtggtaaggcgcgtgaccacctggctgtgctagaccgtaccgaattcgatacggacgtgcgccacgatgccgagatcgtggaacgcgcgctcgtaagcgcggtcattctggccaagatgtcggtgcgcgagacgctggtcacagccatcggccagacggaacccatcgcctttgtgcacctcaaggatacggaggtgcagcgcattgaagaaaacctggagggtgtgcgccgtaacatgttctgcgtgaaaccgctcgaccttaacctggaccggcacgccaacacggcgctggtcaacgccgtcaacaagctcgtgtacacgggccgtctcatcatgaacgtgcgcaggtcttgggaggagctggagcgcaaatgtctggcgcgcattcaggagcgctgcaagctgctggtcaaggagctgcgcatgtgcctttcctttgattccaactactgtcgcaatatcctcaagcacgccgtggaaaacggcgactcggccgacacgctgttggagctgctcatcgaggactttgatatctacgtggacagcttcccacagtcggcgcacacgtttttgggcgcgcgctcgccgtcgttggagtttgacgatgacgccaatctcctctcgctcggcggcggttcggccttctcgtcggtacccaagaaacatgtccccacgcagccgctggacggctggagctggatcgccagtccctggaagggacacaaaccgttccgcttcgaggcccatggttctctggcaccggccgccgaagcccacgctgcccgttcggcggccgtcggctattacgacgaagaggaaaagcgtcgcgagcggcagaaacgggtggacgacgaggtggtgcagcgtgagaaacagcagctgaaggcttgggaggagaggcagcagaacctgcagcaacgtcagcagcaaccaccgcccccggcacgtaaaccgagcgcctcccggaggctctttggctccagtgccgatgaggacgacgacgatgatgatgacgagaaaaacatctttacgcccatcaagaaaccgggaactagcggcaagggcgccgctagtggtggcggtgtttccagcattttcagcggcctgttatcctcgggcagtcagaaaccgaccagcggtcccttgaacatcccgcaacaacaacagcgtcacgcggctttcagtctcgtctccccgcaggtgaccaaggccagcccgggaagggtccgtcgggacagcgcgtgggacgtgaggccgctcacggagaccagaggggatcttttctcgggcgacgaggattccgacagctcggatggctatccccccaaccgtcaagatccgcgtttcaccgacacgctggtggacatcacggataccgagacgagcgccaaaccgcccgtcaccaccgcgtacaagttcgagcaaccgacgttgacgttcggcgccggagttaacgttcctgctggcgccggcgctgccatcctcacgccgacgcctgtcaatccttccacggcccccgctccggccccgacacctaccttcgcgggtacccaaaccccggtcaacggtaactcgccctgggctccgacggcgccgttgcccggggatatgaaccccgccaactggccgcgcgaacgcgcgtgggccctcaagaatcctcacctggcttacaatcccttcaggatgcctacgacttccacggcttctcaaaacaccgtgtccaccacccctcggaggccgtcgactccacgcgccgcggtgacacaaacagcgtctcgggacgccgctgatgaggtttgggctttaagggaccaaactgcagagtcaccggtcgaagacagcgaggaggaagacgacgactcctcggacaccggctccgtcgtcagcctgggacacacaacaccgtcgtccgattacaacaacgacgtcatttcgcctcccagtcagacgcccgagcagtcgacgccgtccagaatacgtaaagctaagttatcgtctccaatgacgacgacatccacgagccagaaaccggtgctgggcaagcgagtcgcgacgccgcacgcgtccgcccgagcgcagacggtgacgtcgacgccggttcagggaaggctagagaaacaggtgtcgggcacgccgtcgacggtacccgccacgctgttgcaacctcaaccggcttcgtctaaaacgacgtcatcaaggaacgtgacttctggcgcgggaacctcttccgcttcttcggctcgacagccgtcagcctcggcgtccgttttgtcgcccacggaggatgatgtcgtgtcccccgccacatcgccgctgtccatgctttcgtcagcctctccgtccccggccaagagtgcccccccgtctccggtgaaaggccggggcagccgcgtcggtgttccttccttgaaacctactttgggcggcaaggcggtggtaggtcgaccgccctcggtccccgtgagcggtagcgcgccgggtcgcctgtccggcagcagccgggccgcctcgaccacgccgacgtatcccgcggtaaccaccgtttacccaccgtcgtctacggccaaaagcagcgtatcgaatgcgccgcctgtggcctccccctccatcctgaaaccgggggcgagcgcggctttgcaatcacgccgctcgacggggaccgccgccgtaggttcccccgtcaagagcacgacgggcatgaaaacggtggctttcgacctatcgtcgccccagaagagcggtacggggccgcaaccgggttctgccggcatggggggcgccaaaacgccgtcggacgccgtgcagaacatcctccaaaagatcgagaagattaagaacacggaggaatag

SEQ ID NO: 16 - protein, 1048 aa, Human cytomegalovirus
MSLQFIGLQRRDVVALVNFLRHLTQKPDVDLEAHPKILKKCGEKRLHRRTVLFNELMLWLGYYRELRFHNPDLSSVLEEFEVRCVAVARRGYTYPFGDRGKARDHLAVLDRTEFDTDVRHDAEIVERALVSAVILAKMSVRETLVTAIGQTEPIAFVHLKDTEVQRIEENLEGVRRNMFCVKPLDLNLDRHANTALVNAVNKLVYTGRLIMNVRRSWEELERKCLARIQERCKLLVKELRMCLSFDSNYCRNILKHAVENGDSADTLLELLIEDFDIYVDSFPQSAHTFLGARSPSLEFDDDANLLSLGGGSAFSSVPKKHVPTQPLDGWSWIASPWKGHKPFRFEAHGSLAPAAEAHAARSAAVGYYDEEEKRRERQKRVDDEVVQREKQQLKAWEERQQNLQQRQQQPPPPARKPSASRRLFGSSADEDDDDDDDEKNIFTPIKKPGTSGKGAASGGGVSSIFSGLLSSGSQKPTSGPLNIPQQQQRHAAFSLVSPQVTKASPGRVRRDSAWDVRPLTETRGDLFSGDEDSDSSDGYPPNRQDPRFTDTLVDITDTETSAKPPVTTAYKFEQPTLTFGAGVNVPAGAGAAILTPTPVNPSTAPAPAPTPTFAGTQTPVNGNSPWAPTAPLPGDMNPANWPRERAWALKNPHLAYNPFRMPTTSTASQNTVSTTPRRPSTPRAAVTQTASRDAADEVWALRDQTAESPVEDSEEEDDDSSDTGSVVSLGHTTPSSDYNNDVISPPSQTPEQSTPSRIRKAKLSSPMTTTSTSQKPVLGKRVATPHASARAQTVTSTPVQGRLEKQVSGTPSTVPATLLQPQPASSKTTSSRNVTSGAGTSSASSARQPSASASVLSPTEDDVVSPATSPLSMLSSASPSPAKSAPPSPVKGRGSRVGVPSLKPTLGGKAVVGRPPSVPVSGSAPGRLSGSSRAASTTPTYPAVTTVYPPSSTAKSSVSNAPPVASPSILKPGASAALQSRRSTGTAAVGSPVKSTTGMKTVAFDLSSPQKSGTGPQPGSAGMGGAKTPSDAVQNILQKIEKIKNTEE